(19) United States
(12) Patent Application Publication
(10) Pub. No.: US 2017/0265780 A1
(43) Pub. Date: Sep. 21, 2017

(54) BAND TYPE SENSOR AND WEARABLE DEVICE HAVING THE SAME

(71) Applicant: LG INNOTEK CO., LTD., Seoul (KR)

(72) Inventors: Sang Young LEE, Seoul (KR); Joon Jae OH, Seoul (KR); Soo Kwang YOON, Seoul (KR); Young Jae LEE, Seoul (KR); Tae Jin LEE, Seoul (KR); Hyun Seok LIM, Seoul (KR); Eun Jung JEON, Seoul (KR)

(21) Appl. No.: 15/528,598

(22) PCT Filed: Dec. 9, 2015
(86) PCT No.: PCT/KR2015/013438

§ 371 Date: May 22, 2017

(30) Foreign Application Priority Data
Dec. 9, 2014 (KR) 10-2014-0176120
Jan. 30, 2015 (KR) 10-2015-0014927
Feb. 6, 2015 (KR) 10-2015-0018548

(51) Int. Cl. A61B 5/11; G06F 3/044; G06F 3/01; A61B 5/00; G06F 1/16

(52) U.S. Cl. CPC A61B 5/11; A61B 5/681; G06F 1/163; G06F 3/011; G06F 3/044

(57) ABSTRACT

A band type sensor includes a substrate and a gesture sensor on the substrate, where the gesture sensor senses the gesture of a user in a capacitive type. And a wearable device having the band type sensor includes a main body, a display part disposed inside the main body, a band connected to the main body, a band type sensor including a touch sensor disposed on the display part and a gesture sensor disposed in the band, and a control unit to perform an operation according to touch and gesture inputs of the user sensed by the band type sensor.

### BACKGROUND

1. Field of the Invention

The embodiment relates to a band type sensor and a wearable device having the same.

2. Description of Related Art

A touch window, which performs an input function through the touch of an image displayed on a display device by an input device, such as a stylus pen or a finger, has been applied to various electronic appliances.

Recently, a touch window has been applied to a wearable device, such as a smart watch or smart glasses, which a user directly wears on his body to be conveniently portable, as well as a device such as a terminal which is used while being directly held by a user.

Such a wearable device is easily portable so that the wearable device has been spotlighted as a next generation device which will be substituted for a general mobile terminal in future.

However, a general touch panel has been applied as a sensor for a user interface applied to the wearable device.

### SUMMARY OF THE INVENTION

When a conventional touch panel for sensing a touch position itself is applied to the wearable device put on a part of the user body such as a body, a neck, a head or a wrist, the merits of the wearable device may be lost.

Therefore, the wearable device requires sensors capable of providing an interface suitable to a user beyond a previous touch window which is simply senses a touch position.

According to the embodiment, a band type sensor may include a substrate and a gesture sensor on the substrate, where the gesture sensor may sense a gesture of a user in a capacitive type.

According to the embodiment, there may be provided a wearable device including the band type sensor thereof.

### Advantageous Effects

According to the embodiment provides a band type sensor which is optimized to a wearable device, so that a new interface can be provided.

### BRIEF DESCRIPTION OF DRAWINGS

The embodiments will be described in detail with reference to the following drawings in which like reference numerals refer to like elements wherein:

FIG. 1 is a substantial perspective view showing a band type sensor according to one embodiment;

FIG. 2 is a view showing a connecting area of FIG. 1;

FIG. 3 is a substantial perspective view showing a band type sensor according to another embodiment;

FIG. 4 is a view showing a connecting area of FIG. 3;

FIG. 5 is a substantial perspective view showing a band type sensor according to still another embodiment;

FIG. 6 is a substantial side view showing a band type sensor according to still another embodiment;

FIG. 7 is a view showing a substrate of a band type sensor according to still another embodiment;

FIG. 8 is a view showing a substrate disposed on a band type sensor according to still another embodiment;

FIG. 9 is a view showing a substrate disposed on a band type sensor according to still another embodiment;

FIG. 10 is a plan view showing a gesture sensor according to still another embodiment;

FIG. 11 is a sectional view taken along ling X-X′ of FIG. 10;

FIG. 12 is a view showing a user wearing a touch device;

FIG. 13 is a sectional view taken along line Y-Y′ of FIG. 12;

FIG. 14 is a view illustrating a capacitance value measured by a gesture sensor in FIG. 12 according to still another embodiment;

FIG. 15 is a view showing a user wearing a touch device;

FIG. 16 is a sectional view taken along line Y-Y′ of FIG. 15;

FIG. 17 is a view illustrating a capacitance value measured by a gesture sensor in FIG. 15 according to still another embodiment;

FIGS. 18 to 24 are plan views showing gesture sensors according to various embodiments;

FIG. 25 is a plane view showing a band type sensor according to still another embodiment;

FIG. 26 is a back view of a band type sensor according to still another embodiment;

FIG. 27 is a sectional view taken along line X-X′ of FIG. 25;

FIGS. 28 to 31 are views showing touch sensors according to various embodiments;

FIGS. 32 and 33 are views showing wearable devices according to embodiments;

FIG. 34 is a block diagram illustrating a wearable device according to an embodiment;

FIG. 35 is a flowchart illustrating a mode change according to whether a wearable device is worn according to an embodiment;

FIG. 36 is a flowchart illustrating a gesture input setting of a wearable device according to an embodiment; and

FIG. 37 is a flowchart illustrating an interface using a band type sensor in a wearable device according to an embodiment.

### DETAILED DESCRIPTION OF THE INVENTION

In the following description of the embodiments, it will be understood that, when a layer (or film), a region, a pattern, or a structure is referred to as being “on” or “under” another substrate, another layer (or film), another region, another pad, or another pattern, it can be “directly” or “indirectly” on the other substrate, layer (or film), region, pad, or pattern, or one or more intervening layers may also be present. Such a position of the layer has been described with reference to the drawings.

In the following description, when a part is connected to the other part, the parts are not only directly connected to each other, but also indirectly connected to each other while interposing another part therebetween. In addition, when a predetermined part “includes” a predetermined component, the predetermined part does not exclude other components, but may further include other components unless otherwise indicated.

The thickness and size of each layer shown in the drawings may be exaggerated, omitted or schematically drawn for the purpose of convenience or clarity. In addition, the size of elements does not utterly reflect an actual size.

Hereinafter, the embodiment will be described with reference to accompanying drawings.

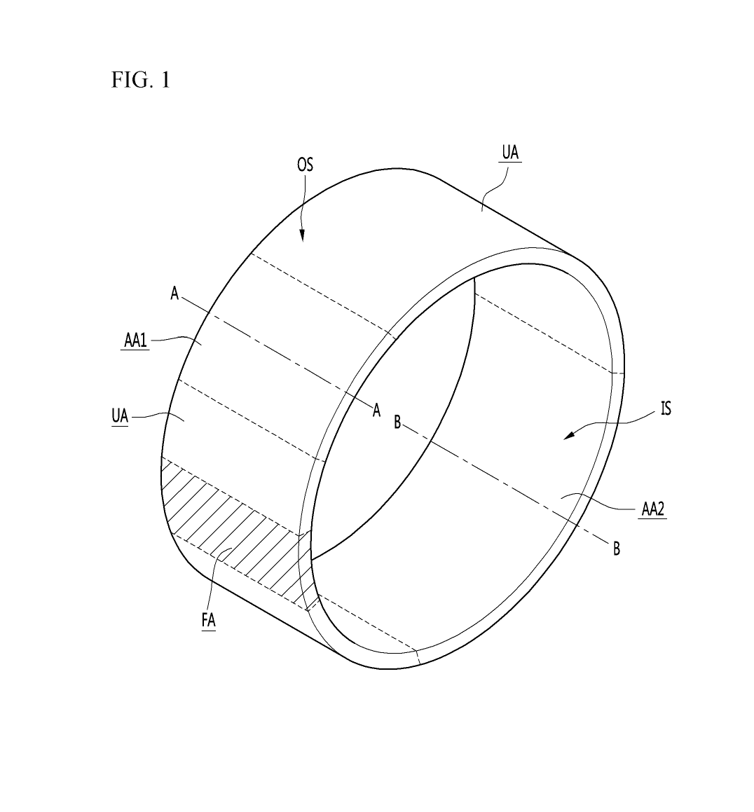

Referring to FIG. 1, a band type sensor may include first and second active areas AA1 and AA2, an unactive area UA and a connecting area FA defined therein.

In detail, the first and second active areas AA1 and AA2 of the band type sensor may be spaced apart from each other. In this case, the unactive area UA may be interposed between the first and second active areas AA1 and AA2 and may have a band shape as shown in FIG. 1.

The connecting area FA may be defined in the unactive area UA. That is, the band type sensor is separated based on the connecting area FA to have a band shape. Both separated ends may be folded or parts of both ends may overlap each other, so that the band type sensor may have a band shape.

Thus, when the band type sensor has the band shape, a user puts the band type sensor on a wearing part and then, both ends of the band are coupled to each other in a band shape in the connecting area FA, such that the user may wear the band type sensor.

When the band type sensor has a band shape, inner surfaces IS of the first and second active areas AA1 and AA2 may face each other.

In addition, the remaining area except for the first and second active areas AA1 and AA2 may be defined as the unactive area UA. In this case, at least a part of the unactive area UA may be defined as the connecting area FA. That is, the connecting area FA may be disposed in the unactive area UA between the first and second active areas AA1 and AA2, such that the first and second active areas AA1 and AA2 may face each other when the band type sensor is worn.

A display may be displayed on the first active area AA1. In addition, a touch sensor for sensing a touch may be disposed on the first active area AA1. For example, a display may be displayed on the first active area AA1 and a GUI capable of controlling the wearable device through a touch may be provided by displaying the display.

The touch sensor may be disposed toward a side of an outer surface OS of the first active area AA1. In detail, the band type sensor may sense the touch input to the outer surface OS of the first active area AA1 which may be seen by a user when both ends of the band type sensor are folded or overlap with each other to be band-shaped.

A gesture sensor capable of sensing a gesture of a user may be disposed on the second active area AA2. For example, when a user wears the band type sensor, at least a part of the second active area AA2 makes contact with the wearing part of the user, so that the gesture of the user may be sensed.

The gesture sensor may be disposed toward a side of an inner surface IS of the second active area AA2. In detail, the band type sensor may sense the gesture of the user through the inner surface IS which makes contact with the wearing part of the user when the user wears the band type sensor.

Before describing the touch sensor and the gesture sensor, various embodiments of the connecting area FA for enabling a user to wear the wearable device will be first described.

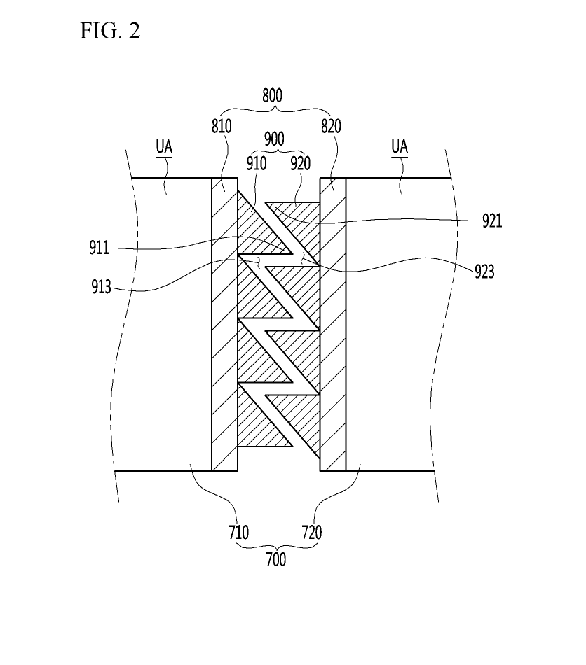

Referring to FIG. 2, a band 700 and a connecting member 900 may be disposed on the connecting area FA of the band type sensor.

The band 700, which is a member constituting an outer shape of the band type sensor, may be formed to surround a wrist when the band 700 is worn around the wrist.

In addition, the band 700 may be formed of a flexible material to be easily worn. For example, the band 700 may be formed of leather, rubber, silicon or synthetic resin.

The band 700 may be disposed to surround a substrate of the band type sensor. That is, the band 700 may surround the gesture sensor disposed on the substrate to protect the gesture sensor.

Thus, band 700 may include the second active area AA2 and the unactive area UA. In advance, the band 700 may include the first active area AA1, but the embodiment is not limited thereto.

The connecting area FA may be disposed in the unactive area UA of the band 700.

The bend 700 may be divided into first and second bands 710 and 720 based on the connecting area FA. The first and second bands 710 and 720 are adhesive to each other or partially overlap each other, so that a band shape is formed. That is, the first band 710 may correspond to the left area of the band 700 and the second band 720 may correspond to the right area of the band 700.

The connecting member 900 may be disposed in the connecting area FA of the band 700.

In detail, the connecting area FA may include a finish member 800 disposed on both ends of the band 700 and the connecting member 900 disposed on the finish member 800. In more detail, the connecting area FA may include a first finish member 810 disposed on one end of the first band 710 and a first connecting member 910 disposed on the first finish member 810.

In addition, the connecting area FA may include a second finish member 820 disposed on one end of the second band 720 and a second connecting member 920 disposed on the second finish member 820.

The first finish member 810 may be detachably coupled to the first band 710. For example, the finish member 800 may be fixed to an end of the first band 710 through a fixing member. When the fixing member for fixing the first finish member 810 is removed, the first finish member 810 may be detached from the first band 710.

Thus, after the first finish member 810 is separated from the band 700, a length of the entire band 700 may be varied by adjusting a length of the first band 710.

The first connecting member 910 may be disposed on the first finish member 810. The first connecting member 910 may include a magnet which has a first magnetic polarity.

The first connecting member 910 may have a concave-convex shape. In detail, the magnet of the first connecting member 910 may have a first protrusion 911 and a first groove 913. For example, the first connecting member 910 may include the first protrusions 911 and the first grooves 913 alternated with each other.

The second finish member 820 may be detachably coupled to the first band 720. For example, the finish member 800 may be fixed to an end of the second band 810 through a fixing member. When the fixing member for fixing the second finish member 820 is removed, the second finish member 820 may be detached from the second band 720. Thus, after the second finish member 820 is separated from the band 700, a length of the entire band 700 may be varied by adjusting a length of the second band 720. However, when the gesture sensor is disposed on the second band 720, the second finish member 820 may not be separated.

The second connecting member 920 may be disposed on the second finish member 820. The second connecting member 920 may include a magnet which has the second magnetic polarity opposite to the first magnetic polarity.

The second connecting member 920 may have a concave-convex shape. In detail, the magnet of the second connecting member 920 may have a second protrusion 921 and a second groove 923. For example, the second connecting member 920 may include the second protrusions 921 and the second grooves 923 alternated with each other.

Since the first and second connecting members 910 and 920 have mutually different polarities, due to the mutually adhering force, the first and second connecting members 910 and 920 may be coupled to each other.

In this case, the concave-convex shapes of the first and second connecting members 910 and 920 may be engaged with each other. That is, when the first and second connecting members 910 and 920 are coupled to each other, the first protrusion 911 of the first connecting member 910 may correspond to the second groove 923 of the second connecting member 920, and the first groove 913 of the first connecting member 910 may correspond to the second protrusion 921 of the second connecting member 920.

The connecting area FA may allow the band type sensor to easily worn and the gesture sensor to be closed to the wearing part of a user when the user wears the band type sensor so that the gesture sensor may be helped to precisely sense a gesture.

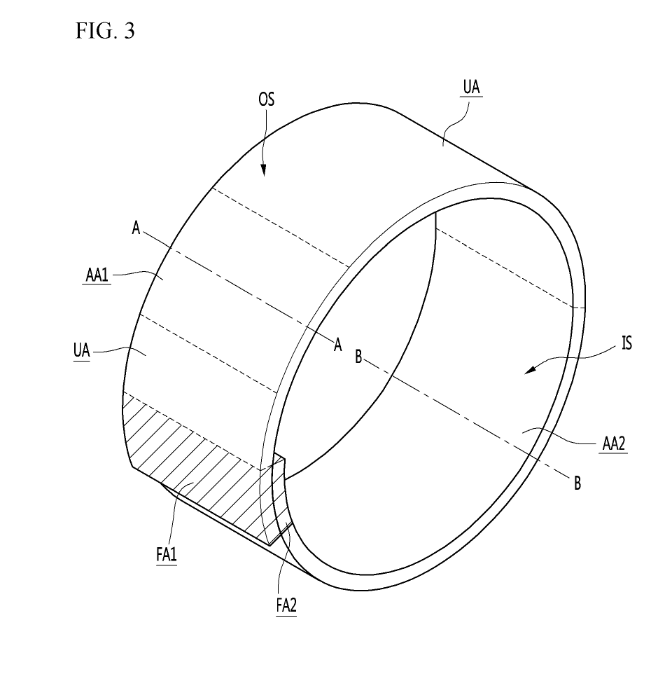

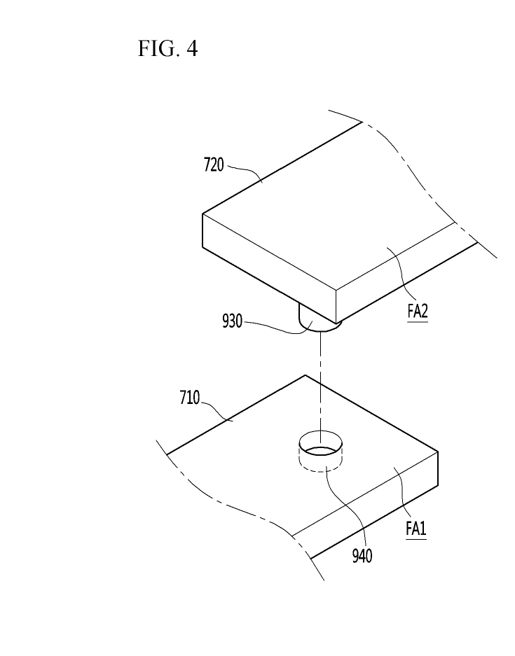

Hereinafter, a band type sensor according to another embodiment will be described with reference to FIGS. 3 and 4. In the following description, the details the same as or similar to those of the above-described embodiment will be omitted and the same references will be assigned to the same elements.

Referring to FIG. 3, a band type sensor may include first and second active areas AA1 and AA2, an unactive area UA and a connecting area FA defined therein.

The band type sensor is separated and coupled based on the connecting area FA. When the band type sensor is coupled, the connecting area FA overlaps the other area. That is, the outer surface of one side of the band type sensor may be disposed on the inner surface IS of the opposite side of the band type sensor.

Thus, when the band type sensor has a shape of a band 700, a user puts the band type sensor on a wearing part and then, both ends of the band are coupled to each other in a band shape in the connecting area FA, such that the user may wear the band type sensor.

When the band type sensor has a band shape, the inner surfaces IS of the first and second active areas AA1 and AA2 may face each other.

Referring to FIG. 4, a band 700 and a connecting member 900 may be disposed on the connecting area FA of the band type sensor.

The band 700, which is a member constituting an outer shape of the band type sensor, may be formed to surround a wrist when the band 700 is worn around the wrist.

The connecting area FA may be disposed on the unactive area UA of the band 700.

The bend 700 may be divided into first and second bands 710 and 720 based on the connecting area FA. The first and second bands 710 and 720 make contact with each other or partially overlap each other, so that a band shape is formed.

In detail, the first band 710 may correspond to the left area of the band 700 and the second band 720 may correspond to the right area of the band 700. The first connecting area FA1 may be disposed on one end of the first band 710, and the second connecting area FA2 may be disposed on one end of the second band 720. The first and second connecting areas FA1 and FA2 may overlap each other.

In detail, when the band 700 is worn on a user to have a band shape, an outer surface OS of the second connecting area FA2 may be disposed on an inner surface IS of the first connecting area FA1. In this case, when the gesture sensor is disposed on the second band 720, the inner surface IS of the band 720 may make contact with the wearing part of the user.

A connecting member 930 and 940 may be disposed in the connecting area FA of the band 700.

*87 At least one groove may be included in the first connecting area FA1 as the connecting member 930 and 940. In detail, at least one groove 940, which is inwardly concaved, may be disposed on the inner surface IS of the first connecting area FA1. If a plurality of grooves 940 exists in the first connecting area FA1, the grooves 940 may be longitudinally disposed while being spaced apart from each other by a constant interval, so that a user may wear the band type sensor after adjusting a diameter of the band 700 corresponding to the wearing part.

In addition, the groove 940 of the first connecting area FA1 may have a first magnetic polarity. That is, a magnet having the first magnetic polarity may be disposed in the groove 940 of the first connecting area FA1.

A protrusion 930 may be included in the second connecting area FA2 as the connecting member 930 and 940. In detail, the protrusion 930 may protrude outwardly from the outer surface OS of the second connecting area FA2.

In addition, the protrusion 930 of the second connecting area FA2 may have the second magnetic polarity opposite to the first magnetic polarity. That is, the protrusion 930 of the second connecting area FA2 may include a magnet having the second magnetic polarity.

A size of the protrusion 930 of the second connecting area FA2 may correspond to the groove 940 of the first connecting area FA1. Thus, the protrusion 930 of the second connecting area FA2 may be closely attached to the inside of the groove 940 of the first connecting area FA1, so that a user may wear the band type sensor.

The connecting area FA according to the embodiment may allow the band type sensor to easily worn and the gesture sensor to be closed to the wearing part of a user, so that the gesture may be precisely sensed.

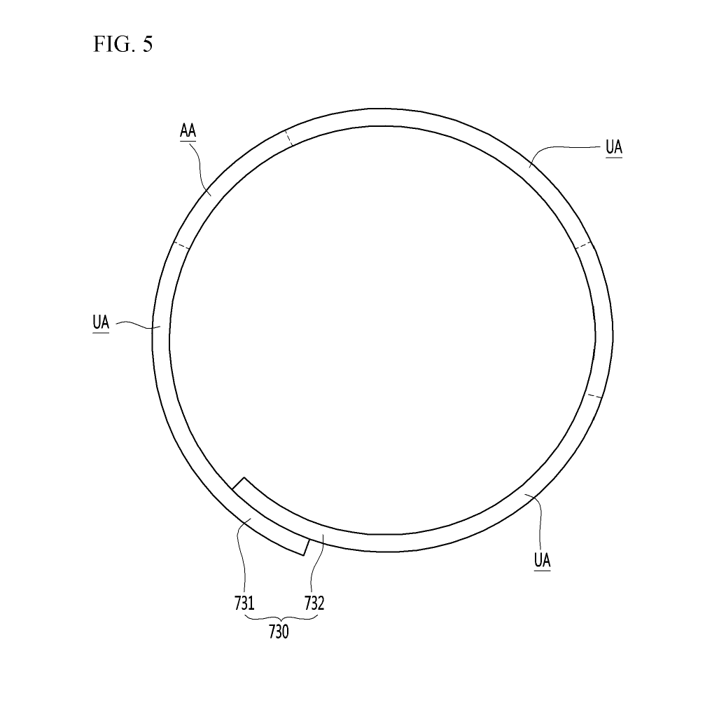

Hereinafter, a band type sensor according to still another embodiment will be described with reference to FIG. 5. In the following description, the details the same as or similar to those of the above-described embodiment will be omitted.

A band type sensor may include first and second active areas AA1 and AA2 and an unactive area UA defined therein.

An outer shape of the band type sensor may be formed by using a band 730.

The band 730, which is a member constituting an outer shape of the band type sensor, may be formed to surround a wrist when the band 730 is worn around the wrist.

The bend 730 may be divided into first and second bands 731 and 732. The first and second bands 731 and 732 make contact with each other or partially overlap each other, so that a band shape is formed. That is, the first band 731 may correspond to the left area of the band 730 and the second band 732 may correspond to the right area of the band 730.

In detail, when the band 730 is worn on a user to have a band shape, an outer surface OS of the unactive area UA of the second band 732 may be disposed on an inner surface IS of the unactive area UA of the first band 731. In this case, the gesture sensor is disposed on the second band 720, so that the inner surface IS of the second band 732 may make contact with the wearing part of the user.

The band 730 may be formed of a flexible material to be easily worn. For example, the band 730 may be formed of leather, rubber, silicon or synthetic resin.

In addition, the band 730 may have a shape memory property.

In addition, the band 730 may have a frame which be formed of shape memory alloy. Such a frame of the band 730 may allow the band 730 to be rolled so that the band 730 has a band shape having a small diameter. A user spreads the band 730 to increase the diameter so that the user may wear the band 730 on a desired body part.

That is, since the band 730 is flexible, the band 730 may be worn on the wearing part in a state that the band 730 has a larger diameter than a diameter of the wearing part and the diameter of the band 730 may be reduced due to the shape memory property so that the band 730 is fastened to the wearing part when the band 730 is worn on the user.

The connecting area FA according to the embodiment may allow the band type sensor to easily worn and the gesture sensor to be closed to the wearing part of a user, so that the gesture may be precisely sensed.

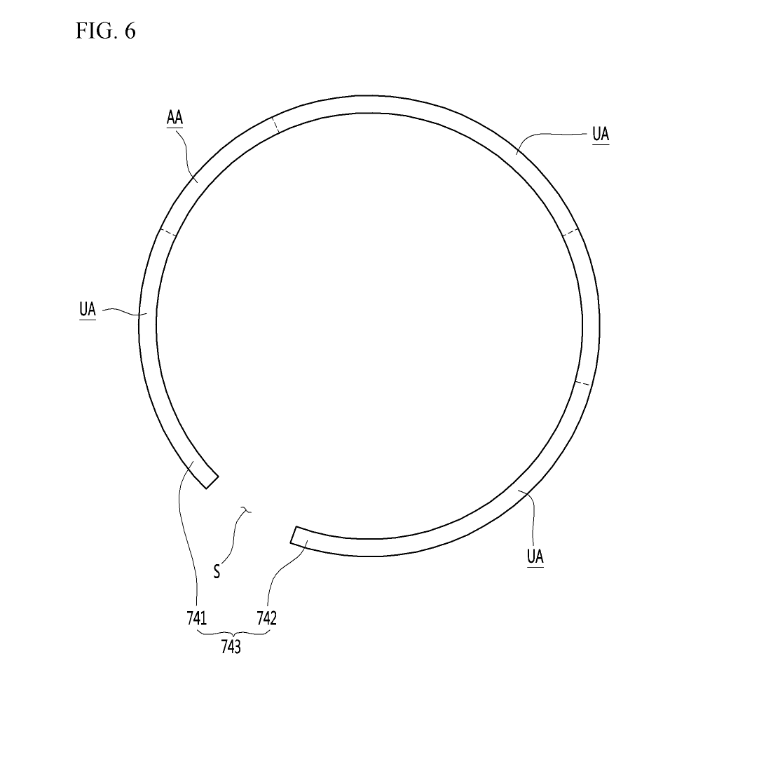

Hereinafter, a band type sensor according to still another embodiment will be described with reference to FIG. 6. In the following description, the details the same as or similar to those of the above-described embodiment will be omitted.

A band type sensor may include first and second active areas AA1 and AA2 and an unactive area UA defined therein.

A band 740, which is a member constituting an outer shape of the band type sensor, may be formed to surround a wrist when the band 740 is worn around the wrist

The bend 740 may be divided into first and second bands 741 and 742. That is, the first band 741 may correspond to the left area of the band 740 and the second band 742 may correspond to the right area of the band 740.

A space may be formed between the first and second bands 741 and 742. That is, even though one ends of the first and second bands 741 and 742 are formed in a band shape, the ends may be separated from each other.

That is, the first and second bands 741 and 742 may be formed in a band shape while including the space therebetween.

In detail, when a user wears the band 740, the one ends of the first and second bands 741 and 742 may make contact with each other or be provided with a small space formed therebetween.

When the band 740 is worn on a user, the one ends of the first and second bands 741 and 742 may be away from each other. That is, the space may be enlarged corresponding to a size of the wearing part of the user.

The band 740 may be formed of a flexible material to be easily worn. For example, the band 740 may be formed of leather, rubber, silicon or synthetic resin.

In addition, the band 740 may have a shape memory property. In addition, the band 740 may have a frame which be formed of shape memory alloy. Such a frame of the band 740 may allow the band 730 to be rolled so that the band 740 has a band shape having a small diameter. A user spreads the band 740 to increase the diameter so that the user may wear the band 730 on a desired body part.

That is, since the band 740 is flexible, the band 740 may be worn on the wearing part in a state that the band 740 has a larger diameter than a diameter of the wearing part and the diameter of the band 740 may be reduced due to the shape memory property so that the band 740 is fastened to the wearing part when the band 730 is worn on the user.

The connecting area FA according to the embodiment may allow the band type sensor to easily worn and the gesture sensor to be closed to the wearing part of a user, so that the gesture may be precisely sensed.

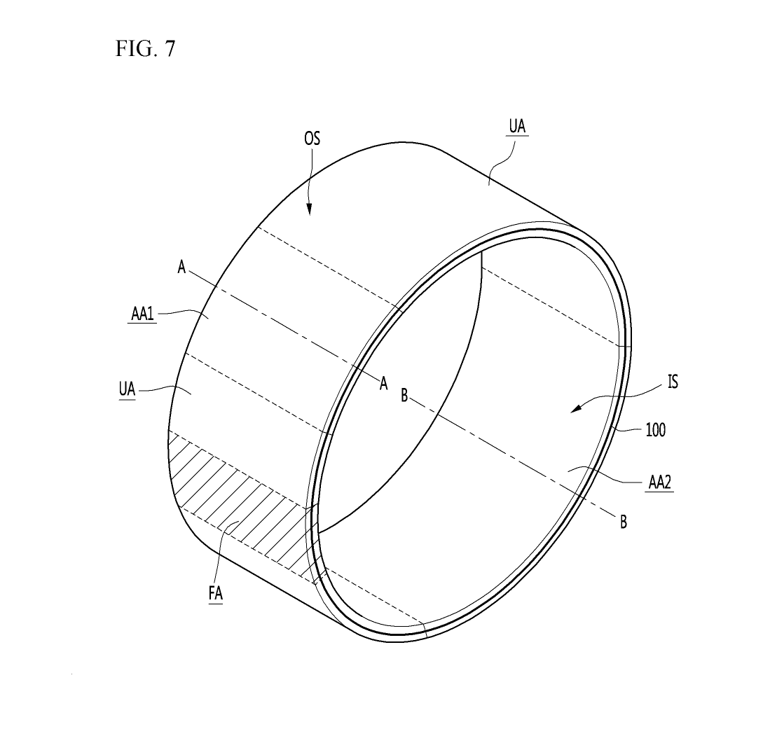

Hereinafter, a substantial configuration of the band type sensor will be described with reference to FIGS. 7 to 9.

Referring to FIGS. 7 to 9, the band type sensor according to one embodiment may include a gesture sensor. In advance, the band type sensor may further include a touch sensor. In more advance, the band type sensor may include a substrate 100 on which the touch sensor and/or the gesture sensor are disposed.

That is, at least one of the touch sensor and the gesture sensor may be disposed.

The substrate 100 may be disposed in the band which is a member constituting an outer shape of the band type sensor.

First, the substrate 100 may have a shape corresponding to the shape of the band type sensor. That is, the substrate 100 may have a band shape. Thus, the substrate 100 may be disposed in at least one of the first and second active areas AA1 and AA2 and the unactive area UA.

According to one embodiment, as shown in FIG. 7, the substrate 100 may be integrally disposed in the first and second active areas AA1 and AA2 and the unactive area UA.

The integrally configured substrate 100 may be disposed in the first and second active areas AA1 and AA2 and the touch sensor and the gesture sensor may be disposed together in a single layer, but the embodiment is not limited thereto.

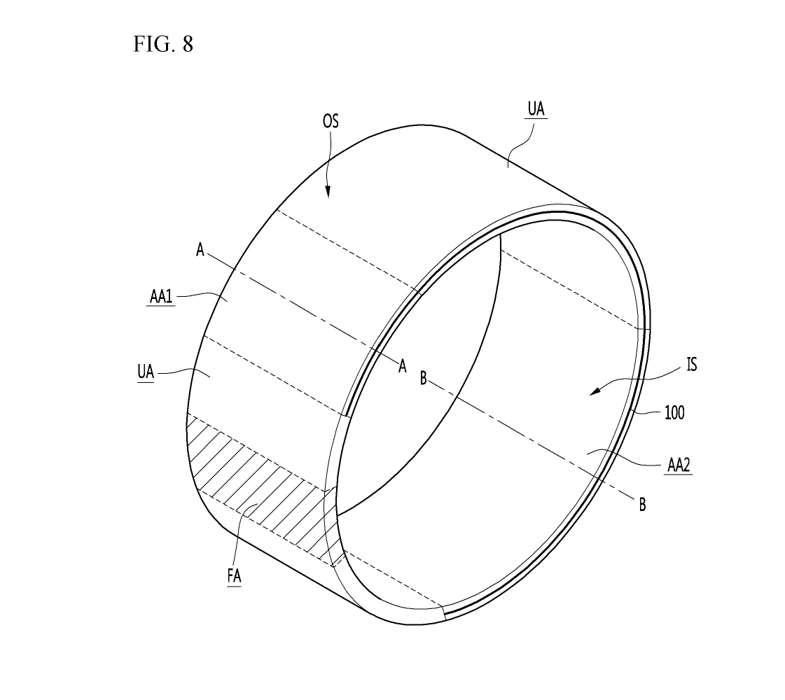

As shown in FIG. 8, a substrate 100 according to another embodiment may correspond to a shape of the band type sensor, but may be partially omitted. For example, the substrate 100 may be disposed in the first and second active areas AA1 and AA2 and a part of the unactive area UA.

The substrate 100 may be disposed in all of the first and second active areas AA1 and AA2 such that the touch sensor and the gesture sensor may be disposed in a single layer, but the embodiment is not limited thereto. According to an embodiment, an unnecessary part of the substrate 100 in the unactive area UA may be omitted so that the cost may be reduced.

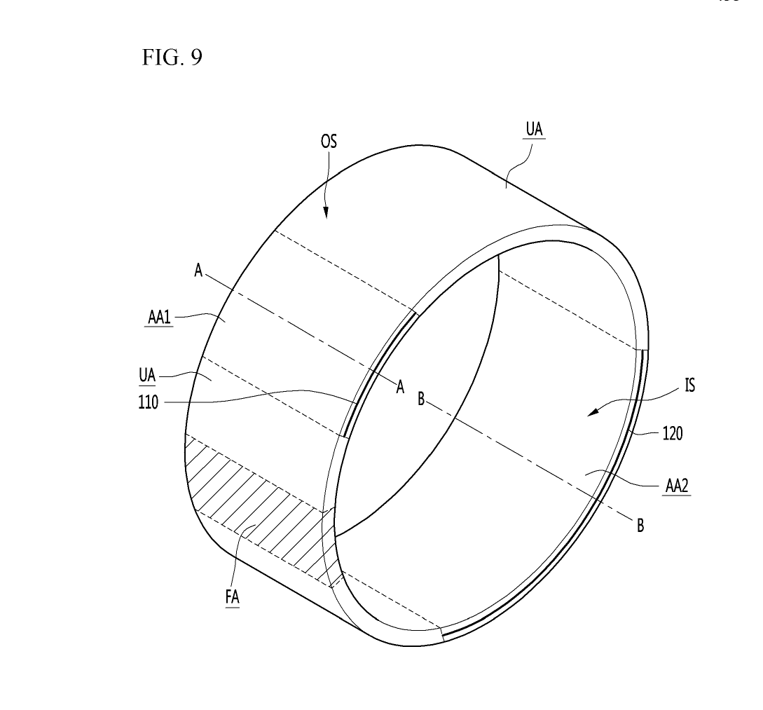

As shown in FIG. 9, a substrate 100 according to still another embodiment may include at least one substrate 100. For example, the substrate 100 may include a second substrate 100 disposed on the second active area AA2. In addition, the substrate 100 may include a first substrate 110 disposed on the first active area AA1.

When the substrate includes the first and second substrates 110 and 120, the first and second substrates 110 and 120 may be spaced apart from each other.

When the substrate 100 is the first or second substrate 110 or 120, the band type sensor may be disposed only on a partial area of the wearable device. For example, when the substrate 100 is the first substrate 110, the band type sensor may be disposed on the area corresponding to a display part of the wearable device. When the substrate 100 is the second substrate 120, the band type sensor may be disposed on the area corresponding to a band part of the wearable device.

According to an embodiment, the band type sensor may include only the gesture sensor.

The entire substrate 100 may be formed of a single material. Alternatively, the substrate 100 may be formed of different kinds of materials in each area. For example, in the substrate 110, the first active area AA1 may be formed of a material different from those of the other areas.

In addition, the substrate 100 may be transparent. In case of the first active area AA1 of the substrate 100, the first active area AA1 may be transparent to show a display. Since it is unnecessary to show the display on the inactive area UA of the substrate 100, the inactive area UA may be opaque, but the embodiment is not limited thereto.

Alternatively, in case as an integrated substrate 100, the entire substrate 100 may be transparent.

In addition, the substrate 100 may be rigid or flexible.

In detail, the first active area AA1 of the substrate 100 may be rigid or flexible. The second active area AA2 and the unactive area UA of the substrate 100 may be flexible for the purpose of convenient wearing.

Alternatively, when the second active area AA2 and the unactive area UA of the substrate 100 are rigid, the second active area AA2 and the unactive area UA may be curved to maintain the band shape.

The substrate 100 may include a glass substrate or a plastic substrate. In detail, the substrate 100 may include chemically tempered/semi-tempered glass, such as soda lime glass or aluminosilicate glass, plastic, such as polyimide (PI), polyethylene terephthalate (PET), poly carbonate (PC), cyclic olefin polymer (COP) film or cyclic olefin copolymer (COC) film, or sapphire.

Meanwhile, the gesture sensor may be disposed on the substrate 100 disposed on the second active area AA2. The gesture of a user may be sensed through the second active area AA2. In detail, when a user wears the band type sensor and makes a predetermined gesture, the gesture may be sensed through the gesture sensor disposed on the second active area AA2.

For example, when a user makes a gesture, a signal may be generated from the gesture sensor disposed on the second active area AA2, so that the gesture of the user may be recognized through the signal.

In detail, the gesture sensor may use an electric signal (EMG) generated from a skeletal muscle as a signal variation due to the gesture of a user. Only, when the EMG signal is used, a sensor electrode for recognizing the EMG may be exposed to an outside of the wearable sensor.

According to an embodiment, a capacitance variation generated by the gesture of a user may be utilized as a gesture recognizing signal. For example, by sensing a variation in capacitance between a user and the sensor electrode of the gesture sensor, the gesture of the user may be recognized.

According to a scheme of sensing a variation in capacitance, it is possible to recognize a variation in a distance between a user and a sensor electrode, so that a gesture of the user may be sensed even though the sensor electrode is spaced apart from the user by a predetermined distance or more. Thus, the gesture may be recognized even when a band is disposed to surround the gesture sensor, so that the gesture sensor may be safely protected and the limitation in design may be overcome.

In addition, it is able to precisely sense a variation in capacitance according to a distance between a user and a sensor electrode, so that the gesture of a user may be precisely recognized.

Hereinafter, a detailed structure of a gesture sensor for sensing a user gesture in a capacitive scheme will be described in detail.

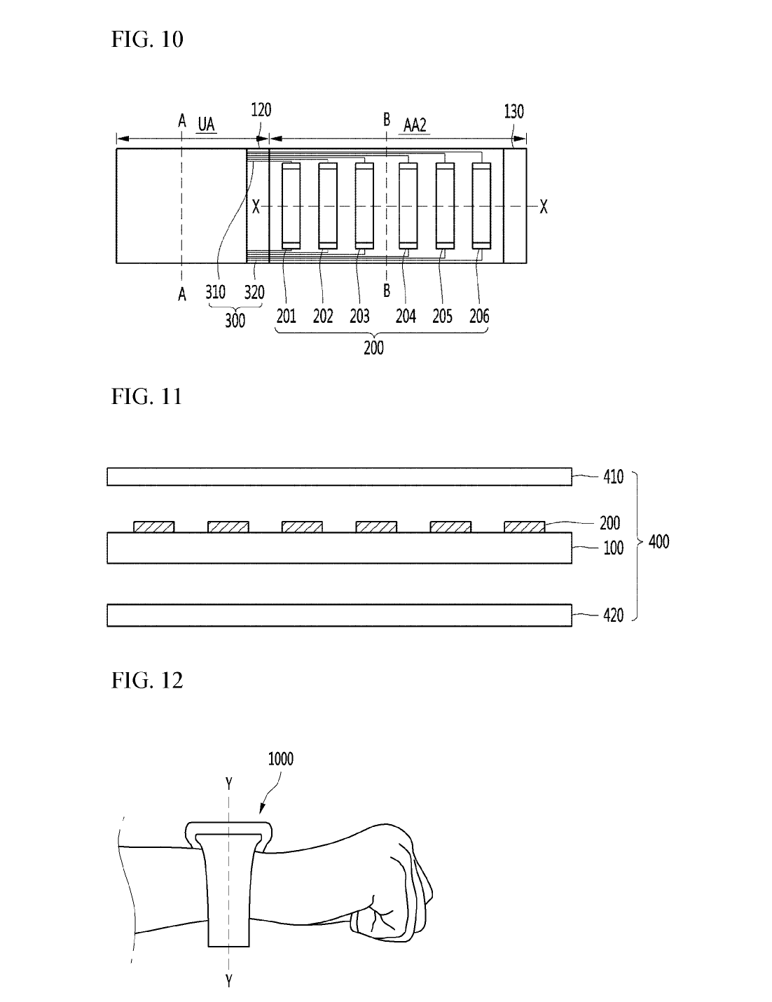

Referring to FIGS. 10 and 11, a gesture sensor according to an embodiment may include a sensor electrode 200 and a sensor wire electrode 300.

First, the sensor electrode 200 may be disposed on one surface of the substrate 100. In detail, the sensor electrode 200 may be disposed on the second active area AA2 of the substrate 100. In more detail, the sensor electrode 200 may be disposed on an inner surface of the second active area AA2. That is, when the substrate 100 has a band shape, the sensor electrode 200 may be disposed on an inner surface of the second active area AA2 of the substrate 100 facing the inner surface of the first active area AA1.

According to an embodiment, the sensor electrode 200 may include at least one electrode pattern.

For example, the sensor electrode 200 may be structured in an array of a plurality of electrode patterns 201 to 206. In detail, the sensor electrode 200 may have bar patterns which are spaced apart from each other by a predetermined interval and repeatedly arrayed to the left and right on the same surface of the substrate 100 to prevent the patterns from making contact with each other. In more detail, the electrode patterns 201 to 206 may be bar patterns which extend vertically and are horizontally arrayed while being spaced part from each other by a constant interval.

FIG. 10 shows the electrode patterns 201 to 206 having a bar shape, but the embodiment is not limited thereto. That is, the sensor electrode 200 may have various shapes capable of sensing whether the band type sensor makes contact with a part of the user body when the band type sensor is worn on the user.

The electrode patterns 201 to 206 disposed as described above measures a variation in capacitance according to the variation in distance between a wearing part of a user and the electrode patterns 201 to 206 when a gesture is input, so that the gesture input of the user may be exactly sensed.

The electrode patterns 201 to 206 of the sensor electrode 200 may be disposed to be bilaterally symmetrical with respect to a reference line of the second active area AA2. In detail, when the number of electrode patterns 201 to 206 is even, the number of electrode patterns 201, 202 and 203 placed left with respect to the reference line may be equal to the number of electrode patterns 204, 205 and 206 placed right with respect to the reference line and the electrode patterns 201 to 206 may be disposed to be bilaterally symmetrical to each other. Alternatively, when the number of electrode patterns is odd, an electrode pattern may be disposed on the reference line and electrode patterns may be disposed to be bilaterally symmetrical with respect to the electrode pattern placed on the reference line.

The sensor electrode 200 may sense a variation in capacitance according to a wearing part of a user and a distance between a contact degree and the contact part, so that the gesture of a user may be sensed.

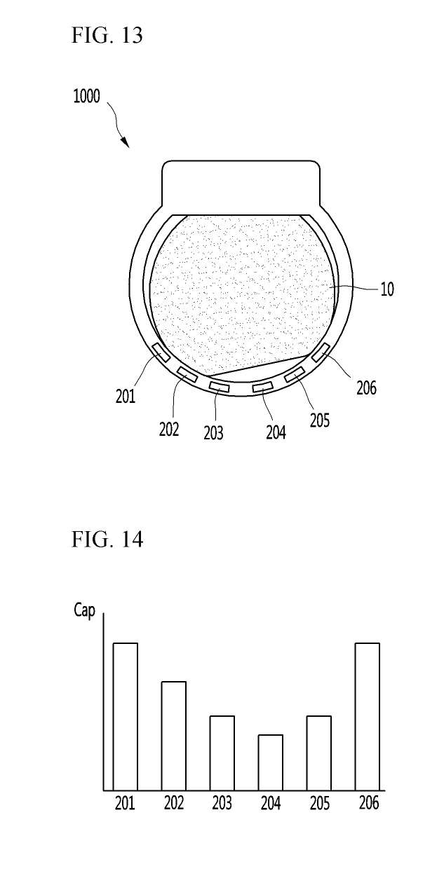

In detail, referring to FIGS. 13 and 16, when a user wearing a wearable device 1000 including the band type sensor on a wrist 10 grasps and opens the hand, the contact area and distance between the wearable device 1000 and the wearing part of the user are changed according to a variation in the human body.

In more detail, while a hand is grasped and opened, the wearing part has a constant shape and is varied, and thus, the contact area and/or the contact position between the wearing part 10 and the wearable device 1000 may be varied.

The sensor electrode 200 may sense the variations in the contact position and/or the contact area by using the variation in capacitance. In detail, when the wearing part 10 makes contact with or is separated from the band of the wearable device in the area corresponding to the gesture sensor, the sensor electrode 200 may sense the variation in capacitance between the sensor electrode 200 and the wearing part 10.

Referring to FIGS. 12 to 14, as the muscle of the wrist 10 is contracted when a user grasps his hand, the gesture sensor may be separated from a part of his wrist. For example, the third to fifth electrode patterns 203 to 205 may be away from the wrist. Thus, the third to fifth electrode patterns 203 to 205 may sense that the capacitance coupled with the wrist is reduced.

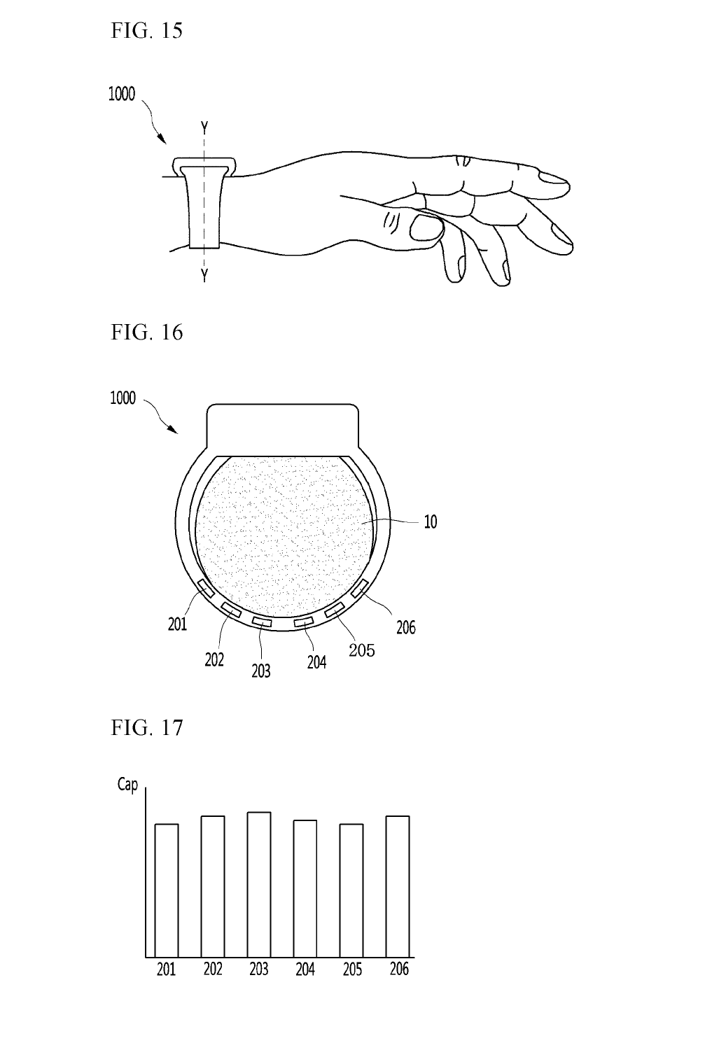

To the contrary, referring to FIGS. 15 to 17, when a user opens his hand, as the muscle of the wrist 10 expands, the entire gesture sensor may make contact with the wrist. Thus, the first to sixth electrode patterns 201 to 206 may sense that the capacitance coupled with the wrist is great. That is, the sensor electrode 200 may recognize the user gesture by measuring the contact area with the user as described above.

As described above, although the actions of grasping and opening a hand as a gesture have been described, the gesture sensor may recognize various gestures by which the contact area between the wearing part 10 and the wearable device 100 is varied. For example, at least one of the actions of stretching his thumb, forefinger, middle finger, ring finger and little finger after a user grasps his hand may be recognized as a gesture. In addition, the muscle of a wrist protruding to the left or right may vary according to the gesture. The gesture sensor may sense the user gesture through the variation of the wrist muscle.

Thus, when a user wears the band type sensor and makes a gesture, all various gestures, which may vary the distance between the wearing part and the band 410 and 420, may be recognized. The variation of the distance may be generated through the variation of a muscle of a user.

A user is able to easily input a signal to the band type sensor through an action of a finger, so that an interface optimized to a wearable device may be provided.

The sensor electrode 200 may sense a variation in capacitance according to a wearing part of a user, a contact area of the band 410 and 420 and a distance by utilizing a self-capacitance scheme and/or a mutual-capacitance scheme.

Since it is possible to recognize the variation in capacitance within a distance between the wearing part and the sensor electrode 200, the sensor electrode 200 may be disposed in the band 410 and 420 of the wearable device 1000.

For example, sensor electrode 200 may sense a gesture of a user through a self-capacitance scheme and/or a mutual-capacitance scheme.

For example, a reference signal may pass through the sensor electrode 200 through a uniform resistance design of the sensor electrode 200. That is, uniform resistance may allow a reference signal to transfer through the sensor electrode 200. A voltage variation may occur due to the variation of the capacitance formed between the wearing part and the sensor electrode 200 when a gesture is input. In this case, the voltage is varied as the capacitance formed between the wearing part and the sensor electrode 200 is varied and the contact position, distance and area may be calculated by calculating the voltage variation with a time. That is, a time difference occurs with respect to a time response according to a voltage variation and thus, the gesture may be sensed by comparing a modified signal with a reference signal. Since the self-capacitance scheme has good sensitivity and enables proximate sensing, even when the wearing part is far away from the sensor electrode 200, the gesture of a user may be exactly sensed.

The sensor electrode 200 may include a conductive material such that electricity flows therethrough. When the band type sensor is disposed on the wearable device 1000, the sensor electrode 200 may disposed in the band 410 and 420, so that it may be no matter that the sensor electrode 200 is opaque.

Thus, the sensor electrode 200 according to an embodiment may include high-conductive metal. For example, the sensor electrode 200 may include at least one of Cr, Ni, Cu, Al, Ag, Mo and the alloy thereof.

However, when the gesture sensor is manufactured together with a touch sensor, for the purpose of convenience of the processing, the gesture sensor may be formed of a material the same as that of an electrode constituting the touch sensor. Since the electrode constituting the touch sensor is required to be transparent, the sensor electrode 200 may include a transparent conductive material.

For example, the sensor electrode 200 may include metallic oxide such as indium tin oxide, indium zinc oxide, copper oxide, tin oxide, zinc oxide, or titanium oxide.

Alternatively, the sensor electrode 200 may include nanowire, photosensitive nanowire film, carbon nanotube (CNT), graphene, or conductive polymer.

In addition, the sensor electrode 200 may include a mesh shape. In detail, the sensor electrode 200 may include a plurality of sub-electrodes which cross each other in a mesh shape.

The sensor wire electrode 300 electrically connected to the sensor electrode 200 may be disposed in the unactive area UA.

The sensor wire electrode 300 may be plural. That is, the sensor wire electrode 300 may include a first sensor wire electrode 310 connected to one end of the sensor electrode 200 and a second sensor wire electrode 320 connected to the opposite end of the sensor electrode 200. Thus, the first sensor wire electrode 310 may be withdrawn to an upper end of the substrate 100. In addition, the second sensor wire electrode 320 may be withdrawn to a lower end of the substrate 100.

After the first sensor wire electrode 310 is withdrawn to the upper end of the substrate 100, the first sensor wire electrode may extend to the first active area AA1 through a first dummy part 101. A printed circuit board 600 may be disposed on the first active area AA1 and the first sensor wire electrode 310 may be connected to the printed circuit board 600.

Likewise, after the second sensor wire electrode 320 is withdrawn to the lower end of the substrate 100, the second sensor wire electrode 320 may extend to the first active area AA1 through the first dummy part 101. In addition, the printed circuit board 600 may be disposed on the first active area AA1 and the second sensor wire electrode 320 may be connected to the printed circuit board 600.

A process which can measure and calculate a variation in capacitance transferred through the sensor electrode 200 may be disposed on the printed circuit board 600.

In this case, the processor may be connected to the touch sensor disposed on the first active area AA1, so that the processor may measure and calculate the variation in capacitance transferred through the touch sensor. Thus, the band type sensor according to an embodiment may drive the touch sensor and the gesture sensor through a single processor.

However, the embodiment is not limited to the above, and the band type sensor may include an additional printed circuit board 600 for the gesture sensor and an additional processor.

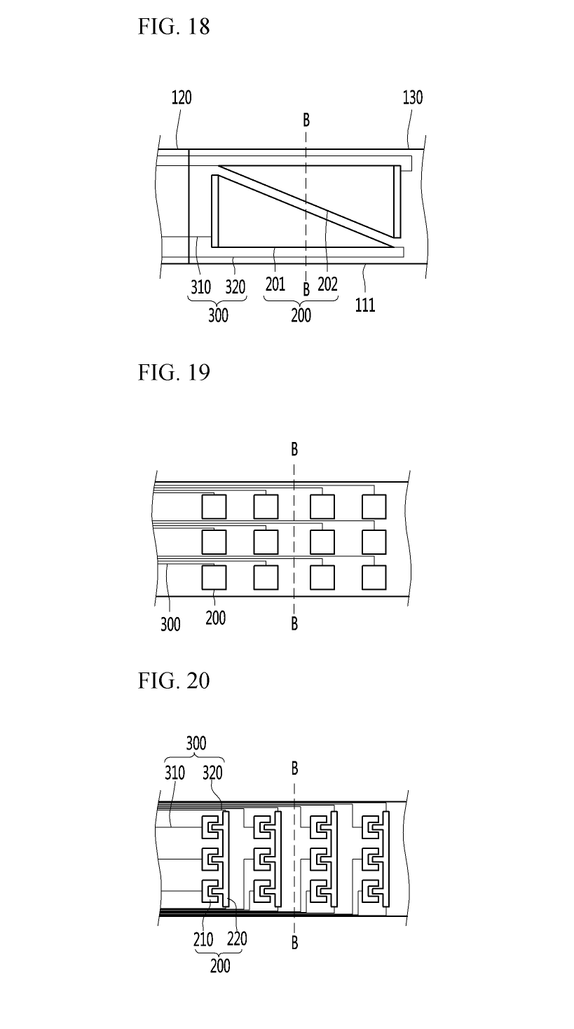

Hereinafter, a band type sensor according to another embodiment will be described with reference to FIGS. 18 to 24. In the following description, the details the same as or similar to those of the gesture sensor according to the above-described embodiment will be omitted and the same references will be assigned to the same elements.

As described above, the sensor electrode 200 of the gesture sensor may have various shapes. First, referring to FIG. 18, the gesture sensor according to an embodiment may include a sensor electrode 200 and a sensor wire electrode 300.

First, the sensor electrode 200 may be disposed on one surface of the substrate 100. In detail, the sensor electrode 200 may be disposed on the unactive area UA of the substrate 100.

The sensor electrode 200 may include a plurality of electrode patterns 201 and 202 disposed in mutually different rows. The electrode patterns 201 and 202 may have widths varying in a longitudinal direction in order to sense a position of the substrate 100 in the longitudinal direction. That is, the electrode pattern 201 and 202 may be a triangular pattern. In detail, the electrode patterns 201 and 202 may be right-angled triangular patterns, where two right-angled triangular patterns may be disposed on the substrate 200 to allow the hypotenuses to face each other.

The sensor electrodes 201 and 202 of the sensor electrode 200 may be disposed to be bilaterally symmetrical with respect to reference line B-B of the unactive area UA. In detail, the electrode patterns 201 and 202 may be disposed to allow the center of a base side of a rectangular pattern to be placed on reference line B-B.

The sensor electrode 200 may sense the capacitance according to a degree of contact between the body of a user and the sensor electrode 200. In detail, when a user makes a gesture, the human body varies into a predetermined shape, and thus, the contact area and/or the contact position between the human body and the wearable device may be varied. The sensor electrode 200 may sense the variations in the contact position and/or the contact area based on the variation in capacitance.

The sensor wire electrode 300 electrically connected to the sensor electrode 200 may be disposed in the unactive area UA. The sensor wire electrode 300 may be plural.

A process which can measure and calculate a variation in capacitance transferred through the sensor electrode 200 may be disposed on the printed circuit board 600.

If the processor is connected even to the touch sensor, the processor may measure and calculate the variation in capacitance transferred through the touch sensor.

Referring to FIG. 19, the gesture sensor according to an embodiment may include a sensor electrode 200 and a sensor wire electrode 300.

First, the sensor electrode 200 may be disposed on one surface of the substrate 100. In detail, the sensor electrode 200 may be disposed on the second active area AA2 of the substrate 100. The sensor electrode 200 may be constructed in an array of electrode patterns.

The sensor electrode 200 may include the electrode patterns disposed in mutually different columns and rows. That is, the electrode patterns may be disposed in a matrix form.

The electrode patterns may include a bar pattern, a rhombus pattern, a triangular pattern, a rectangular pattern and a random pattern.

The electrode patterns of the sensor electrode 200 may be disposed to be bilaterally symmetrical with respect to a reference line of the second active area AA2.

In addition, the sensor electrode 200 may sense the capacitance according to a degree of contact between the body of a user and the sensor electrode 200. In detail, when a user makes a gesture, the human body varies into a predetermined shape and is varied, and thus, the contact area and/or the contact position between the human body and the wearable device. The sensor electrode 200 may sense the variations in the contact position and/or the contact area based on the variation in capacitance.

The sensor wire electrode 300 electrically connected to the sensor electrode 200 may be disposed in the unactive area UA. In detail, each of the electrode patterns may be individually connected to the sensor wire electrode 300.

The sensor wire electrode 300 may extend to the first active area AA1 while passing through a first dummy part 101. A printed circuit board 600 may be disposed on the first active area AA1 and the sensor wire electrode 300 may be connected to the printed circuit board 600.

A process which can measure and calculate a variation in capacitance transferred through the sensor electrode 200 may be disposed on the printed circuit board 600. The processor may be connected to the touch sensor disposed on the active area, so that the processor may measure and calculate the variation in capacitance transferred through the touch sensor.

Referring to FIG. 20, the gesture sensor according to still another embodiment may include a sensor electrode 200 and a sensor wire electrode 300.

First, the sensor electrode 200 may be disposed on one surface of the substrate 100. In detail, the sensor electrode 200 may be disposed on the second active area AA2 of the substrate 100. In more detail, when the substrate 100 has a band shape, the substrate 100 may be disposed on the second active area AA2 which includes a reference line overlapping with the first active area AA1.

The sensor electrode 200 according still another embodiment may include first and second sensor electrodes 210 and 220. Each of the sensor electrodes 200 may include a plurality of electrode patterns.

For example, the first sensor electrode 210 may include a plurality of electrode patterns which may be disposed in a matrix form.

The sensor electrode 220 may include a plurality of electrode patterns, and the electrode pattern of the second sensor electrode 220 may be spaced apart from the electrode patterns of the first sensor electrode 210.

The electrode patterns of the sensor electrode 200 may be disposed to be bilaterally symmetrical with respect to a reference line of the second active area AA2.

The sensor electrode 200 may sense the capacitance according to a degree of contact between the body of a user and the sensor electrode 200. In detail, when a user makes a gesture, the body has a constant shape and is varied, and thus, the contact area and/or the contact position between the body and the wearable device may be varied.

The sensor electrode 200 may sense the variations in the contact position and/or the contact area by using the variation in capacitance. In detail, when the human body makes contact with or is separated from the wearable sensor in the area corresponding to the gesture sensor, the sensor electrode 200 may sense the variation in capacitance between the sensor electrode 200 and the human body.

The sensor wire electrode 300 electrically connected to the sensor electrode 200 may be disposed in the second active area AA2. In detail, each of the electrode patterns may be individually connected to the sensor wire electrode 300.

The sensor wire electrode 300 may extend to the first active area AA1 while passing through a first dummy part 101. A printed circuit board 600 may be disposed on the first active area AA1 and the sensor wire electrode 300 may be connected to the printed circuit board 600.

A process which can measure and calculate a variation in capacitance transferred through the sensor electrode 200 may be disposed on the printed circuit board 600. The processor may be connected to the touch sensor disposed on the active area, so that the processor may measure and calculate the variation in capacitance transferred through the touch sensor.

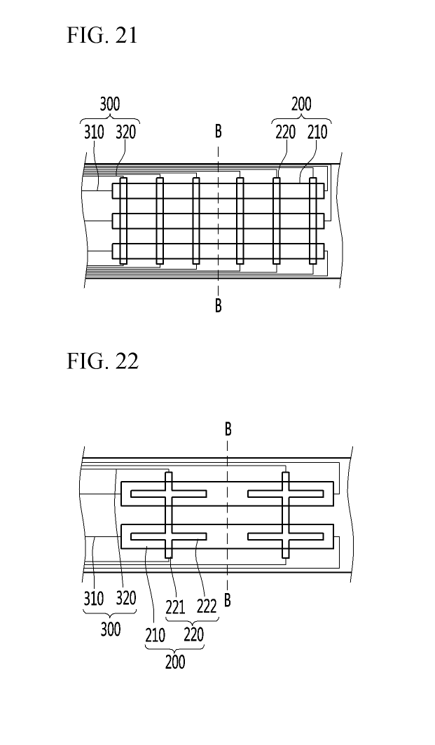

Referring to FIG. 21, the gesture sensor according to still another embodiment may include a sensor electrode 200 and a sensor wire electrode 300.

First, the sensor electrode 200 may be disposed on one surface of the substrate 100. In detail, the sensor electrode 200 may be disposed on the second active area AA2 of the substrate 100. In more detail, when the substrate 100 has a band shape, the substrate 100 may be disposed on the second active area AA2 which includes a reference line overlapping with the first active area AA1.

In addition, the sensor electrode 200 may include first and second sensor electrodes 210 and 220. In detail, the first sensor electrode 210 having first directionality and the second electrode 220 having second directionality may be disposed on a single substrate 100 and an insulating member may be interposed between the first and second sensor electrodes 210 and 220 such that the first and second electrodes 210 and 220 are prevented from making contact with each other. In addition, the sensor wire electrode 300 connected to the sensor electrode 200 may be disposed on the substrate 100.

Since the capacitance induced between the first and second sensor electrodes 210 and 220 is varied when the body of a user makes contact with the gesture sensor, a gesture of the user may be sensed.

The sensor wire electrode 300 electrically connected to the sensor electrode 200 may be disposed on the substrate 100. In detail, a first sensor wire electrode 310 extending from the first sensor electrode 210 and a second sensor wire electrode 320 extending from the second sensor electrode 220 may be disposed on the substrate 100.

The first and second sensor wire electrode 310 and 320 may extend to the first active area AA1 while passing through a first dummy part 101. A printed circuit board 600 may be disposed on the first active area AA1 and the sensor wire electrode 300 may be connected to the printed circuit board 600.

A process which can measure and calculate a variation in capacitance transferred through the sensor electrode 200 may be disposed on the printed circuit board 600. The processor may be connected to the touch sensor disposed on the active area, so that the processor may measure and calculate the variation in capacitance transferred through the touch sensor.

Referring to FIG. 22, the gesture sensor according to still another embodiment may include a sensor electrode 200 and a sensor wire electrode 300.

First, the sensor electrode 200 may be disposed on one surface of the substrate 100. In detail, the sensor electrode 200 may be disposed on the second active area AA2 of the substrate 100. In more detail, when the substrate 100 has a band shape, the substrate 100 may be disposed on the second active area AA2 which includes a reference line overlapping with the first active area AA1.

The sensor electrode 200 may include a first sensor electrode 210 extending in a first direction and a second sensor electrode 220 extending in a second direction.

The first and second sensor electrodes 210 and 220 may be disposed on one surface of the substrate 100. In detail, first and second sensor electrodes 210 and 220 may be disposed on the same surface of the substrate 100. That is, the first and second sensor electrodes 210 and 220 may be disposed on the same surface of the substrate 100 while being spaced apart from each other, such that the first and second sensor electrodes 210 and 220 are prevented from making contact with each other. For example, any electrodes may not be formed on the area of the first sensor electrode 210 overlapping the second sensor electrode 220.

The second sensor electrode 220 may include a branch electrode and the first sensor electrode 210 may surround the branch electrode. Thus, the capacitance coupled between the first and second sensor electrodes 210 and 220 may be increased.

Since the capacitance induced between the first and second sensor electrodes 210 and 220 is varied when the body of a user makes contact with the gesture sensor, a gesture of the user may be sensed.

The sensor wire electrode 300 electrically connected to the sensor electrode 200 may be disposed on the substrate 100. In detail, a first sensor wire electrode 310 extending from the first sensor electrode 210 and a second sensor wire electrode 320 extending from the second sensor electrode 220 may be disposed on the substrate 100.

The first and second sensor wire electrode 310 and 320 may extend to the active area while passing through a first dummy part 101. A printed circuit board 600 may be disposed on the active area and the sensor wire electrode 300 may be connected to the printed circuit board 600.

A process which can measure and calculate a variation in capacitance transferred through the sensor electrode 200 may be disposed on the printed circuit board 600. The processor may be connected to the touch sensor disposed on the active area, so that the processor may measure and calculate the variation in capacitance transferred through the touch sensor.

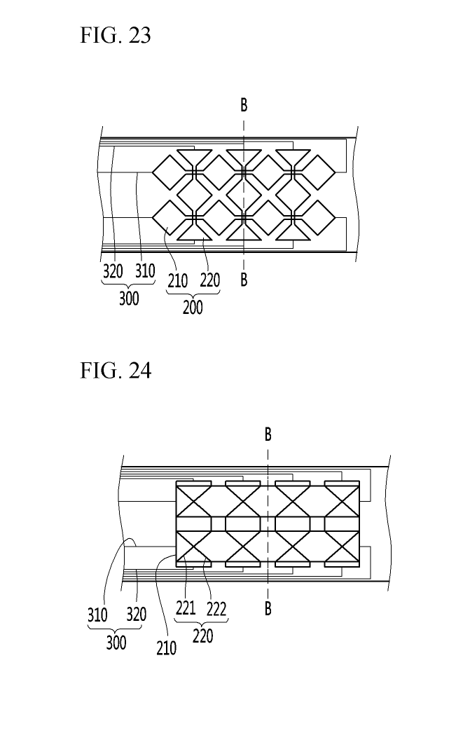

Referring to FIG. 23, the gesture sensor according to still another embodiment may include a sensor electrode 200 and a sensor wire electrode 300.

First, the sensor electrode 200 may be disposed on one surface of the substrate 100. In detail, the sensor electrode 200 may be disposed on the second active area AA2 of the substrate 100. In more detail, when the substrate 100 has a band shape, the substrate 100 may be disposed on the second active area AA2 which includes a reference line overlapping with the first active area AA1.

According to an embodiment, the sensor electrode 200 may include first and second sensor electrodes 210 and 220. In detail, the first sensor electrode 210 having first directionality and the second electrode 220 having second directionality may be disposed on a single substrate 100 and an insulating member may be interposed between the first and second sensor electrodes 210 and 220 such that the first and second electrodes 210 and 220 are prevented from making contact with each other. In addition, the sensor wire electrode 300 connected to the sensor electrode 200 may be disposed on the substrate 100.

In this case, according to an embodiment, the first and second sensor electrodes 210 and 220 may further include a rhombus pattern. In detail, the rhombus patterns of the first and second sensor electrodes 210 and 220 may cross each other. An insulating member may be further disposed in the area in which the first and second sensor electrodes 210 and 220 overlap each other.

Since the capacitance induced between the first and second sensor electrodes 210 and 220 is varied when the body of a user makes contact with the gesture sensor, a gesture of the user may be sensed. The rhombus patterns of the first and second sensor electrodes 210 and 220 increase the capacitance induced between the first and second sensor electrodes 210 and 220, so that the gesture may be more precisely sensed.

The sensor wire electrode 300 electrically connected to the sensor electrode 200 may be disposed on the substrate 100. In detail, a first sensor wire electrode 310 extending from the first sensor electrode 210 and a second sensor wire electrode 320 extending from the second sensor electrode 220 may be disposed on the substrate 100.

The first and second sensor wire electrode 310 and 320 may extend to the first active area AA1 while passing through a first dummy part 101. A printed circuit board 600 may be disposed on the first active area AA1 and the sensor wire electrode 300 may be connected to the printed circuit board 600.

A process which can measure and calculate a variation in capacitance transferred through the sensor electrode 200 may be disposed on the printed circuit board 600. The processor may be connected to the touch sensor disposed on the active area, so that the processor may measure and calculate the variation in capacitance transferred through the touch sensor.

Referring to FIG. 22, the gesture sensor according to still another embodiment may include a sensor electrode 200 and a sensor wire electrode 300.

First, the sensor electrode 200 may be disposed on one surface of the substrate 100. In detail, the sensor electrode 200 may be disposed on the second active area AA2 of the substrate 100. In more detail, when the substrate 100 has a band shape, the substrate 100 may be disposed on the second active area AA2 which includes a reference line overlapping with the first active area AA1.

In addition, the sensor electrode 200 may include first and second sensor electrodes 210 and 220. In detail, the first sensor electrode 210 having first directionality and the second electrode 220 having second directionality may be disposed on a single substrate 100 and an insulating member may be interposed between the first and second sensor electrodes 210 and 220 such that the first and second electrodes 210 and 220 are prevented from making contact with each other. In addition, the sensor wire electrode 300 connected to the sensor electrode 200 may be disposed on the substrate 100.

Since the capacitance induced between the first and second sensor electrodes 210 and 220 is varied when the body of a user makes contact with the gesture sensor, a gesture of the user may be sensed. The branch electrode of the first sensor electrode 210 may increase the capacitance coupled with the second sensor electrode 220, so that the variation of the contact with the body may be more precisely sensed.

The sensor wire electrode 300 electrically connected to the sensor electrode 200 may be disposed on the substrate 100. In detail, a first sensor wire electrode 310 extending from the first sensor electrode 210 and a second sensor wire electrode 320 extending from the second sensor electrode 220 may be disposed on the substrate 100.

The sensor wire electrode 300 may extend to the first active area AA1 while passing through a first dummy part 101. A printed circuit board 600 may be disposed on the first active area AA1 and the sensor wire electrode 300 may be connected to the printed circuit board 600.

A process which can measure and calculate a variation in capacitance transferred through the sensor electrode 200 may be disposed on the printed circuit board 600. The processor may be connected to the touch sensor disposed on the active area, so that the processor may measure and calculate the variation in capacitance transferred through the touch sensor.

The band type sensor according to an embodiment may be formed on the substrate only with the gesture sensor. The band type sensor may be applied to a smart band. The smart band may not require any touch sensors because the smart band do not have any displays (or may include an additional touch screen) and may be utilized for an exercise-aid apparatus or a health diagnosis device.

Meanwhile, the gesture and touch sensors may be formed integrally with each other.

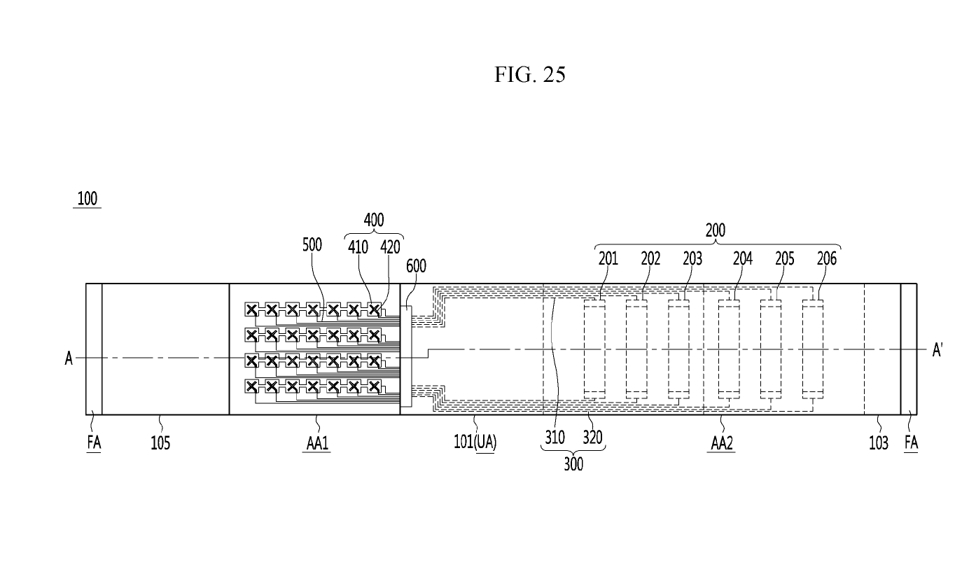

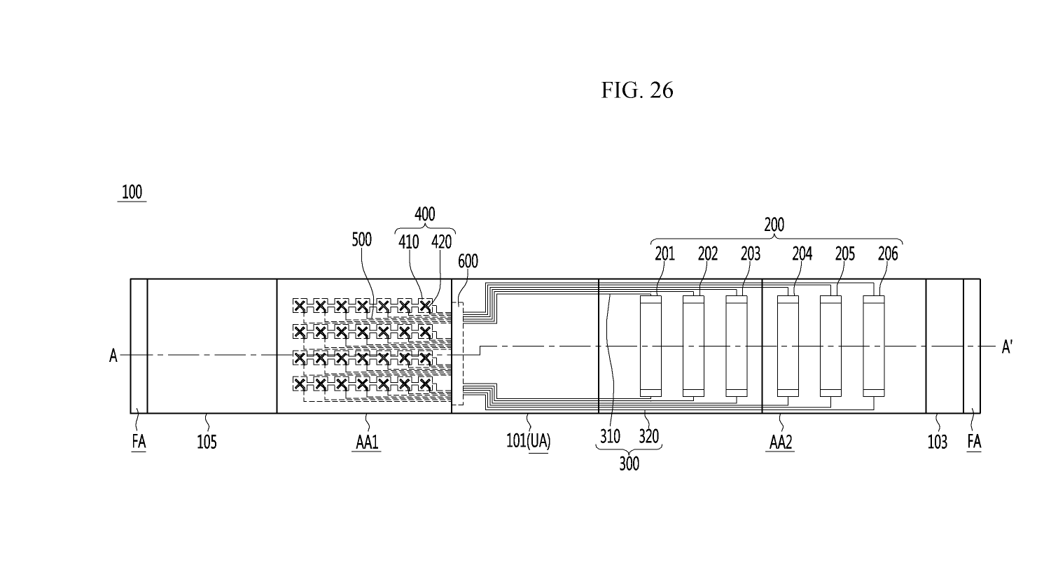

Hereinafter, a band type sensor in which gesture and touch sensors are formed integrally with each other will be described with reference to FIGS. 25 to 29.

Referring to FIGS. 25 to 27, the touch sensor may be disposed on the substrate 100 disposed on the first active area AA1. In detail, the touch sensor may be disposed on an outer surface of the substrate 100 of the first active area AA1. That is, when the touch sensor is disposed on one surface of the substrate 100, the gesture sensor may be disposed on an opposite surface of the substrate 100. The touch sensor may sense a touch position of a user through an input device (for example, a finger).

For example, the input device (for example, a finger) touches the first active area AA1, the capacitance between the input device and the sensing electrode 400 of the touch sensor is varied so that the part on which the variation occurs may be detected as the touch point.

That is, both the gesture sensor for recognizing a user gesture and the touch sensor for sensing a touch point of a user may be disposed on the substrate 100 of the band type sensor according to an embodiment.

When the gesture and touch sensors are formed at the same time, the gesture and touch sensor may sense a user gesture and a user touch based on a variation in capacitance. Since the variation in capacitance may be processed by a single processor, the user gesture and touch may be sensed by using a single processor. Thus, the cost of the band type sensor may be reduced.

When the gesture and touch sensors are together disposed, the unactive area UA may be disposed between the first and second active areas AA1 and AA2 in order to prevent the interference between the gesture and touch sensors. In detail, the first dummy part 101 may be disposed on the unactive area UA of the substrate 100 between the first active area AA1 on which the touch sensor is disposed and the second area AA2 on which the gesture sensor is disposed. In more detail, the first dummy part 101 may be disposed between one ends of the first and second active areas AA1 and AA2.

Although the first dummy part 101 is disposed to allow the unactive area UA to be horizontally spaced apart from the active areas, the unactive area UA may be spaced apart from the active areas in three dimensions. That is, when the substrate 100 includes first and second substrates 110 and 120, the first dummy part 101 may correspond to the space between the first and second substrates 110 and 120.

In addition, a second dummy part 103 and 105 may be disposed on the opposite ends of the first and second active areas AA1 and AA2.

A connecting part of the wearable device may be disposed on the second dummy part 103 and 105.

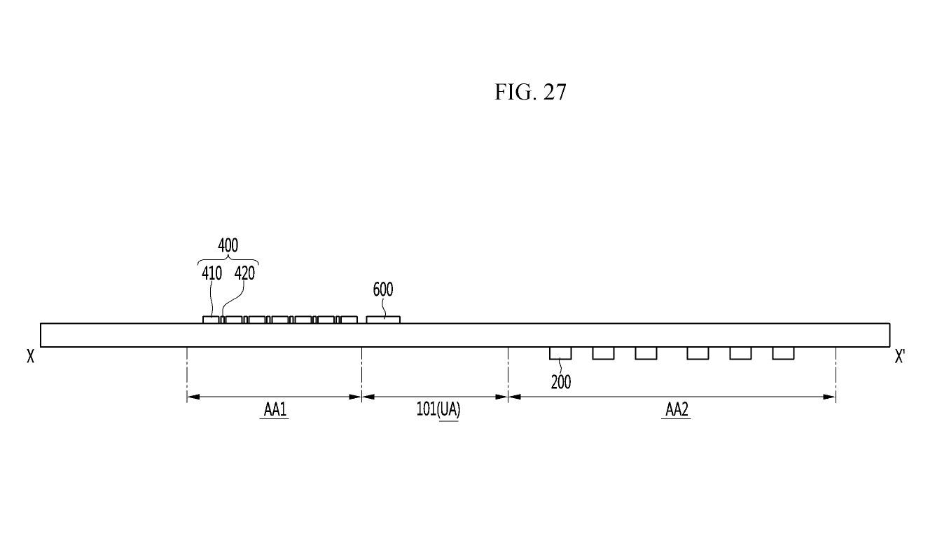

As shown in FIG. 27, according to an embodiment, the touch and gesture sensors may be disposed on another surface of the substrate 100. In this case, the printed circuit board 600 may be disposed on the same surface as the touch sensor. The substrate 100 may have a hole such that the substrate 100 is connected to the printed circuit board 600. That is, the sensor wire electrode 300 may be connected to the printed circuit board 600 while passing through the hole of the substrate 100.

Alternatively, one part of the printed circuit board 600 may be disposed on one surface of the substrate 100 on which the wire electrode 400 of the touch sensor is disposed and another part of the printed circuit board 600 may be disposed on the opposite surface of the substrate 100 on which the sensor wire electrode 300 of the gesture sensor is disposed. Thus, the wire electrode 400 and the sensor wire electrode 300 may be connected to the printed circuit board 600 without forming any additional holes on the substrate 100. In this case, the printed circuit board 600 may be disposed to surround one side surface of the substrate 100.

A process which can measure and calculate a variation in capacitance transferred through the sensor electrode 200 may be disposed on the printed circuit board 600. The processor may be connected to the touch sensor disposed on the first active area AA1, so that the processor may measure and calculate the variation in capacitance transferred through the touch sensor. Thus, the band type sensor according to an embodiment may drive the touch sensor and the gesture sensor through a single processor

However, the embodiment is not limited to the above, and the band type sensor may include an additional printed circuit board 600 for the gesture sensor and an additional processor.

Meanwhile, the touch sensor may be disposed on the first active area AA1. In detail, the touch sensor may be disposed on an outer surface of the first active area AA1.

In addition, the touch sensor may include a sensing electrode 400, a wire electrode 500 and a printed circuit board 600.

The sensing electrode 400 may be disposed on the first active area AA1 of the substrate 100. In detail, the sensing electrode 400 may be disposed on the outer surface of the first active area AA1 of the substrate 100. In more detail, the sensing electrode 400 may make direct contact with the outer surface of the first active area AA1 of the substrate 100.

The sensing electrode 400 may include first and second sensing electrodes 410 and 420.

The first and second sensing electrodes 410 and 420 may be disposed on one surface of the substrate 100. In detail, the first and second sensing electrodes 410 and 420 may be disposed on the same surface of the substrate 100. That is, the first and second sensing electrodes 410 and 420 may be spaced apart from each other such that the first and second sensing electrodes 410 and 420 may be prevented from making contact with each other on the same surface of the substrate 100.

Since the first and second sensing electrodes 410 and 420 according to an embodiment may be formed on the same one surface so that any additional substrates 100 are not required, the touch sensor may have a thin thickness and the cost may be reduced.

The sensor electrode 400 may sense a touch point by utilizing a self-capacitance scheme and/or a mutual-capacitance scheme.

For example, the first and second sensing electrodes 410 and 420 may be capacitive-coupled to each other so that the touch position may be sensed through mutual capacitance. The touch sensing of the capacitive scheme may be multi-touch sensible and may enable a touch position to be exactly sensed.

At least one of the first and second sensing electrodes 410 and 420 may include a transparent conductive material to prevent electricity from flowing therethrough without interfering light transmission. At least one 400 of the first and second sensor electrodes 410 and 420 may include metallic oxide such as indium tin oxide, indium zinc oxide, copper oxide, tin oxide, zinc oxide or titanium oxide.

Alternatively, at least one 400 of the first and second sensor electrodes 410 and 420 may include nanowire, photosensitive nanowire film, carbon nanotube (CNT), graphene, conductive polymer or a mixture thereof.

In addition, at least one of the first and second sensing electrodes 410 and 420 may include various metals. For example, at least one 400 of the first and second sensing electrode 410 and 420 may include at least one of Cr, Ni, Cu, Al, Ag, Mo, Au, Ti and the alloy thereof.

The sensing electrode 400 may include a mesh shape. In detail, the sensing electrode 400 may include a plurality of sub-electrodes which cross each other in a mesh shape.

In detail, the sensing electrode 400 may include mesh lines LA by the sub-electrodes crossing each other in a mesh shape and a mesh opening part OA between the mesh lines LA. In this case, a line width of the mesh line LA may be in the range of about 0.1 μm to about 10 μm. If the line width of the mesh line LA is less than about 0.1 μm, the mesh line LA may not be fabricated. If the line width of the mesh line LA exceeds about 10 μm, a sensing electrode pattern may be visually recognized from an outside, so that the visibility may be degraded. In addition, the line width of the mesh line LA may be in the range of about 1 μm to about 5 μm. Preferably, the line width of the mesh line LA may be in the range of about 1.5 tin to about 3 μm.

The mesh opening OA may be formed in various shapes. For example, the mesh opening OA may have various shapes such as a polygonal shape including a rectangular shape, a diamond shape, a pentagonal shape or a hexagonal shape, or a circular shape. In addition, the mesh opening may be formed in a regular or random shape.

As the sensing electrode 400 has a mesh shape, the pattern of the sensing electrode 400 may not be viewed on the active or unactive areas AA1, AA2 or UA. That is, even when the sensing electrode 400 is formed of metal, the pattern may not be viewed. In addition, even when the sensing electrode 400 is applied to a large-size touch sensor, the resistance of the sensing electrode 400 may be reduced.

The wire electrode 500 may be disposed on the substrate 100. In detail, the wire electrode 500 may be disposed on the same surface as that of the sensing electrode 400.

The wire electrode 500 may be disposed on the first active area AA1 of the substrate 100. In detail, the wire electrode 500 may be disposed on the first active area AA1 of the substrate 100 and extend to be connected to the printed circuit board 600.

The wire electrode 500 may include a material the same as or similar to that of the sensing electrode 400 described above.

Alternatively, the wire electrode 500 may include heterogeneous materials. In detail, the wire electrode 500 may include a transparent conductive material and/or a non-transparent conductive material. For example, the wire electrode 500 disposed on the first active area AA1 may include a transparent conductive material and the wire electrode 500 disposed on the unactive area UA may include a non-transparent material.

The printed circuit board 600 may be disposed on at least one of the first active area AA1 and an adjacent unactive area UA. For example, the printed circuit board 600 may be disposed on the unactive area UA making contact with the first active area AA1.

The printed circuit board may include a processor. Thus, the touch signal sensed by the sensing electrode 400 may be transmitted to the processor through the wire electrode 500.

The printed circuit board 600 may be flexible. That is, the printed circuit board 600 may be flexible printed circuit board (FPCB).

The printed circuit board 600 may be connected to the sensor wire electrode 300.

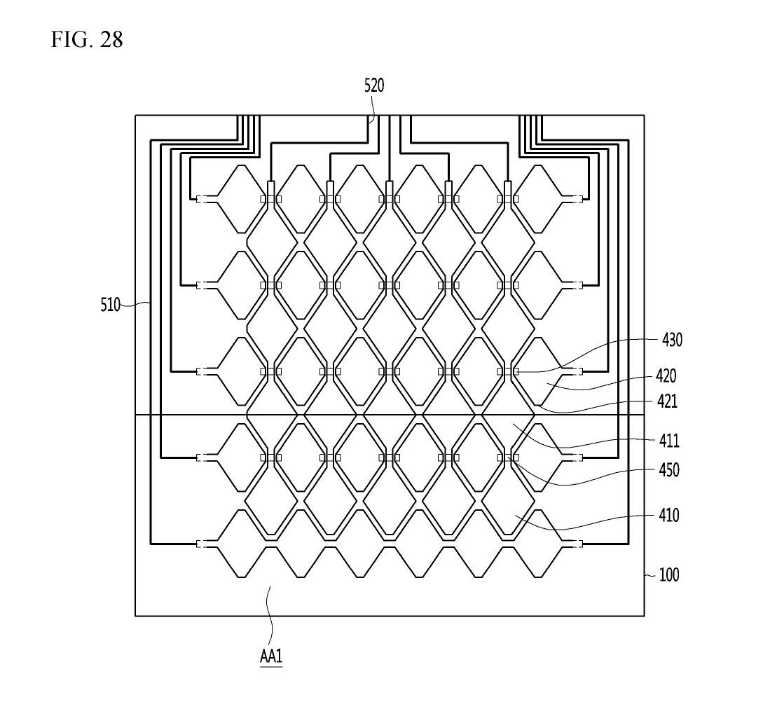

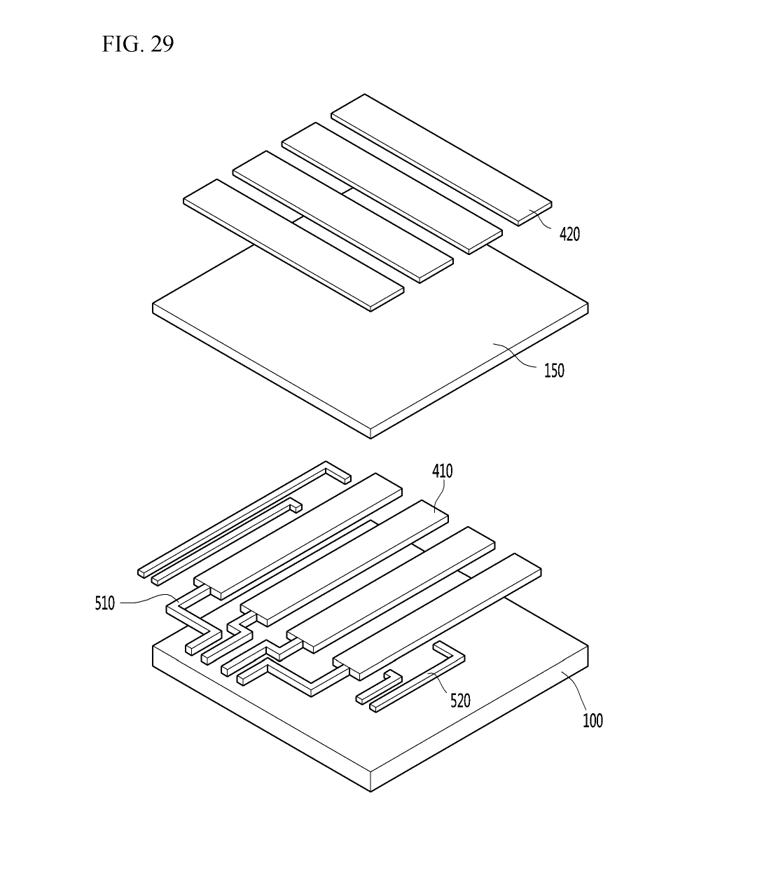

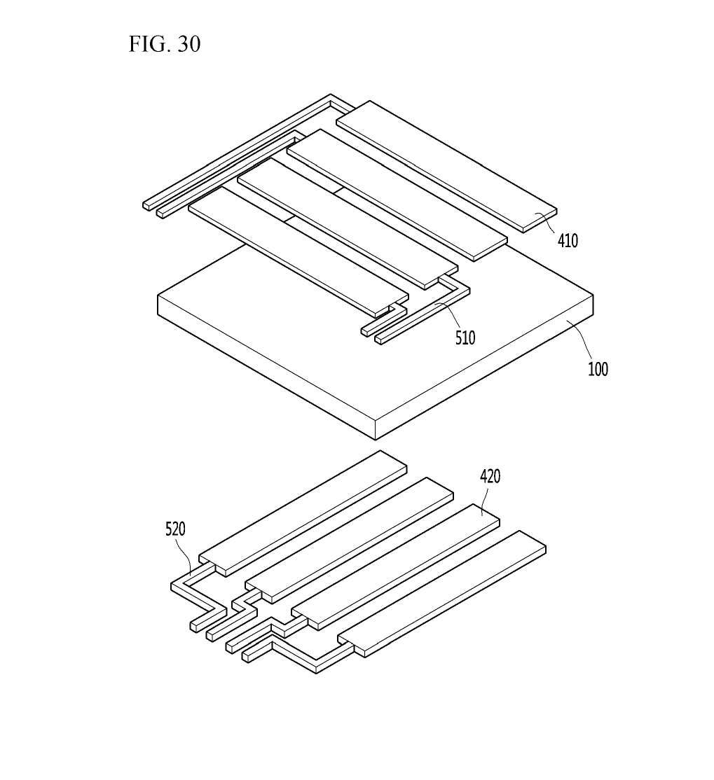

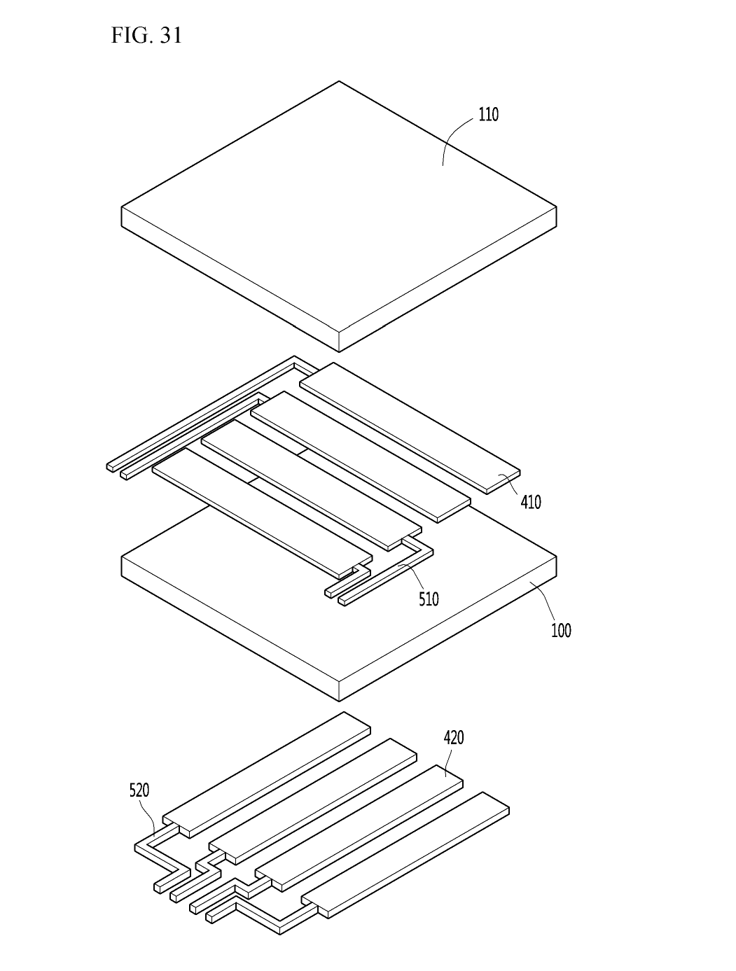

Hereinafter, various embodiments of the touch sensor will be described with reference to FIGS. 28 to 31. In the following description, the details the same as or similar to those of the touch sensor according to the above-described embodiment will be omitted and the same references will be assigned to the same elements.

Referring to FIG. 28, a touch sensor according to another embodiment may include a substrate 100 and a sensing electrode 400. The sensing electrode 400 may include first and second sensing electrodes 410 and 420.

The first and second electrodes 410 and 420 may be disposed on the same surface of the substrate 100. The first and second electrodes 410 and 420 may be spaced apart from each other on one surface of the substrate 100.

In more detail, the first sensing electrode 410 may extend in the first direction on the first active area AA1. The first sensing electrode 410 may make direct contact with the substrate 100. In addition, the second sensing electrode 420 may extend in the second direction on the first active area AA1. In detail, the second electrode 420 may extend in the second direction different from the first direction and make direct contact with the substrate 100. That is, the first and second sensing electrodes 410 and 420 may make direct contact with the same surface of the substrate 100 and may extend in mutually different directions on the same surface of the substrate 100.

The first and second sensing electrodes 410 and 420 may be disposed on the substrate 100 while being insulated from each other.

A bridge electrode 430 may be provided on one surface of the substrate 100 on which the sensing electrode 400 is disposed. For example, the bridge electrodes 430 may be arranged in a bar shape. In detail, the bridge electrodes 430 may be spaced apart from each other by a predetermined interval on the first active area AA1 while being disposed in the bar shape.

An insulating material 450 may be provided on the bridge electrode 430. In detail, the insulating material 450 may be partially formed on the bridge electrode 430, so that a part of the bridge electrode 430 may be covered with the insulating material 450. For example, when the bridge electrode 430 is formed in a bar shape, the insulating material 450 may be formed on the bridge electrode 430 except for one end and the opposite end of the bridge electrode 430, that is, both ends of the bridge electrode 430.

The first sensing electrodes 410 may are connected to each other and may extend on the insulating material 450. For example, the first sensing electrodes 410 extending in the first direction may be disposed to be connected to each other on the insulating material 450.

In addition, the second sensing electrode 420 may be disposed to be connected to the bridge electrode 430. In detail, the second sensing electrodes 420 spaced apart from each other may be connected to the bridge electrodes 430. Thus, the second sensing electrodes 420 may extend in the second direction.

Thus, the first and second sensing electrodes 410 and 420 may be electrically connected to each other without being short-circuited to each other due to the bridge electrodes and the insulating material.

Since the sensing electrode 400 may be formed in a single layer, the touch sensor may be thinly formed. In addition, since any additional substrates 100 are not required, the cost may be reduced.

Referring to FIG. 29, a touch sensor according to still another embodiment may include a substrate 100, a sensing electrode 400, a wire electrode 500, a printed circuit board 600 and an intermediate layer 150. The sensing electrode 400 may include a first sensing electrode 410 extending in the first direction and a second sensing electrode 420 extending in the second direction different from the first direction.

In detail, the first sensing electrode 410 may be disposed on the first active area AA1 of the substrate 100. In addition, the first wire electrode 500 connected to the first sensing electrode 410 and the second wire electrode 500 connected to the second sensing electrode 420 may be disposed on the first active area AA1 of the substrate 100.

An intermediate layer 150 may be disposed on the first active area AA1 of the substrate 100. In this case, a sectional area of the intermediate layer 400 may be different from a sectional area of the first active area AA1. For example, the sectional area of the intermediate layer 150 may be smaller than that of the first active area AA1. Accordingly, when the intermediate layer may cover the first sensing electrode 410 disposed on the first active area AA1 and may not cover the wire electrode 500.

The intermediate layer 150 may be directly disposed on the first active area AA1. That is, the intermediate layer 150 may be formed by directly a dielectric material on a top surface of the first active area AA1 of the substrate 100 on which the first sensing electrode 410 is disposed.

The second sensing electrode 420 may be disposed on the intermediate layer 150 and the first and second sensing electrodes 410 and 420 may be insulated from each other through the intermediate layer 150.

The intermediate layer 150 may include a material different from the substrate 100. For example, intermediate layer 150 may include a dielectric material.

For example, the intermediate layer 150 may include an insulating group including halogen compound of alkali metal or alkali earth metal, such as LiF, KCl, CaF2, or MgF2, or fused silica, such as SiO2, SiNX, etc.; a semiconductor group including InP or InSb; transparent oxide used for semiconductor or dielectric substance including In compound, such as ITO or IZO, mainly used for a transparent electrode, or transparent oxide used for semiconductor or dielectric substance, such as ZnOx, ZnS, ZnSe, TiOx, WOx, MoOx, or ReOx; an organic semiconductor group including Alq3, NPB, TAPC, 2TNATA, CBP or Bphen; and a low-K material such as silsesquioxane or a derivative ((H—SiO3/2)n) thereof, methylsilsesquioxane (CH3-SiO3/2)n), porous silica or porous silica doped with fluorine or carbon atoms, porous zinc oxide (ZnOx), cyclized-perfluoropolymer (CYTOP) or a mixture thereof.

In addition, the intermediate layer 150 may have visible ray transmittance of about 75% to 99%.

In this case, a thickness of the intermediate layer 150 may be less than that of the substrate 100. In detail, the thickness of the intermediate layer 150 may be about 0.01 to about 0.1 times that of the substrate 100. For example, the thickness of the substrate 100 may be about 0.1 mm and the thickness of the intermediate layer 150 may be about 0.001 mm, but the embodiment is not limited thereto.

Referring to FIG. 30, a touch sensor according to still another embodiment may include a substrate 100, a sensing electrode 400, a wire electrode 500 and a printed circuit board 600.

In detail, a first sensing electrode 410 extending in one direction and a first wire electrode 500 connected to the first sensing electrode 410 may be disposed on one surface of the substrate 100. A second sensing electrode 420 extending in a direction different from the one direction and a second wire electrode 500 connected to the second sensing electrode 420 may be disposed on the other surface of the substrate 100, that is, a surface opposite to the one surface of the substrate 100.

Referring to FIG. 31, a touch sensor according to still another embodiment may include a cover substrate 110, a substrate 100, a sensing electrode 400, a wire electrode 400, and a printed circuit board 600.

The cover substrate 110 may be disposed on the substrate 100. The cover substrate 110 may be rigid or flexible. For example, the cover substrate 110 may include glass or plastic. In detail, the cover substrate 110 may include plastic such as polyethylene terephthalate (PET) or polyamide (PI), or sapphire.

In addition, a portion of the cover substrate 110 may be curved with a partial curved surface. That is, a portion of the cover substrate 110 may have a flat surface, and another portion of the cover substrate 110 may be curved with a curved surface. In detail, an end portion of the cover substrate 110 may be curved with a curved surface or may be curved or bent with a surface having a random curvature.

The cover substrate 110 and the substrate 100 may be bonded to each other through an adhesive layer. For example, the cover substrate 110 and the substrate may be bonded to each other through an optical clear adhesive (OCA).

The sensing electrode 400 may be disposed on the cover substrate 110 and the substrate 100. For example, the first sensing electrode 410 may be disposed on the cover substrate 110 and the second sensing electrode 420 may be disposed on the substrate 100.

That is, the first sensing electrode 410 may be disposed on the cover substrate 110. In addition, the second sensing electrode 420 may be additionally disposed on the substrate 100.

In addition, the wire electrode 500 may include a first wire electrode 510 connected to the first sensing electrode 410 and a second wire electrode 520 connected to the second sensing electrode 420. The first wire electrode 510 may be disposed on the cover substrate 110 and the second wire electrode 500 may be disposed on the substrate 100.

The band type sensors according to embodiments described above may be applicable to all of a neckband type device worn on the neck of a user, a headset type device worn on the head of a user, and a wearable device such as a watch type device worn on the wrist of a user.

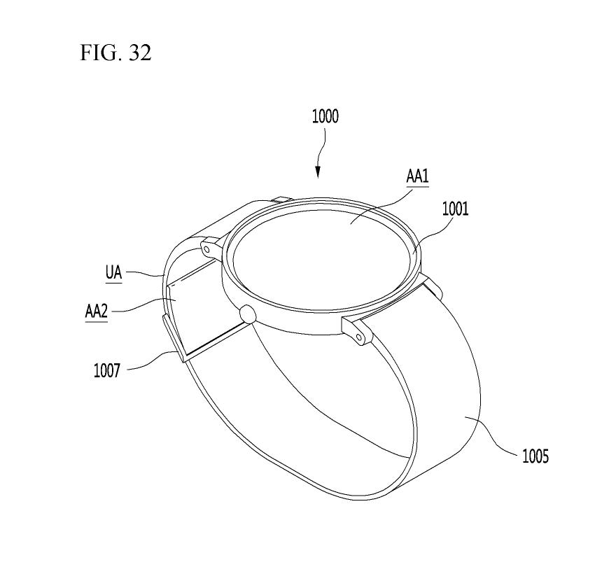

Hereinafter, a wearable device, especially, a watch type mobile terminal will be described with reference to FIG. 32.

Referring to FIG. 32, the wearable device 1000 may include a main body 1001, a display unit, a band type sensor and a band 1005.

First, the main body 1001 may include a case constituting an outer appearance. The case of the main body 1001 may have an inner space receiving various kinds of electronic components. In this case, the main body 100 may be divided into first and second cases for forming the inner space.

The display unit may be disposed on a front surface of the main body 1001 to display information. A touch sensor of the band type sensor may be disposed on the display unit and include a touch screen.

Thus, the wearable device 1000 may provide an interface to a user through the touch screen.

The band 1005 may be connected to the main body 1001. The band 1005 may be formed to be worn on a wrist while surrounding the wrist. The band 1005 may be formed of a flexible material to be easily worn. For example, the band 700 may be formed of leather, rubber, silicon or synthetic resin.

A gesture sensor of the band type sensor may be disposed in the band 1005. That is, the gesture sensor of the band type sensor may be disposed in the band 1005.

The band 1005 may include a fastener 1007 for fastening the band 1005. The fastener 1007 may be implemented by using a buckle, a snap-fit hook structure or Velcro® (trade mark) and may include an elastic section or material. In the drawing, an example of the fastener 107 implemented in a buckle form is proposed. The fastener 107 may be disposed on the band 1005 adjacent to a side of the first active area AA1 so that the gesture sensor may be exposed o user's hand.

As another example, the band 1005 may have an integral member of an elastic material instead of the fastener 1007 such that a user can wear the band 1005 on his wrist.

According to an embodiment, the fastener 1007 may be replaced with the connecting part of the embodiments described above.

In addition, the band 1005 may be detachably constructed on the main body 1001. Thus, the band 1005 may be changed into various types of bands 1005 according to user preference.

The gesture sensor of the band type sensor may be disposed in the band 1005. When a user wears the wearable device 1000 and makes a gesture, the gesture sensor may sense the gesture.

Thus, the wearable device 1000 may provide a gesture interface to allow a user to control a device through a gesture input.

That is, the wearable device 100, to which the band type sensor having the gesture sensor is applied, may provide the gesture interface to a user to improve user convenience.

In addition, the wearable device 100, to which the band type sensor having the gesture and touch sensors is applied, may provide the touch and gesture interfaces at the same time. Thus, the interface suitable to the wearable device 1000 for user convenience may be provided so that the user convenience may be more improved.

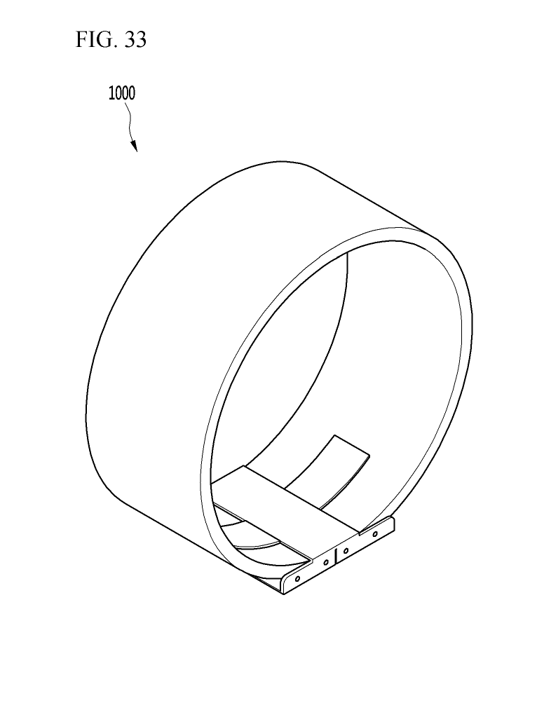

Referring to FIG. 33, the wearable device may be a smart band. In this case, the smart band may not require any additional touch screen. That is, although the smart band has a simple display (such as an LED light), the smart band may not have any displays. The smart band may be utilized for an exercise-aid apparatus or a health diagnosis device and the band type sensor may be applied to the smart band to provide the gesture interface.

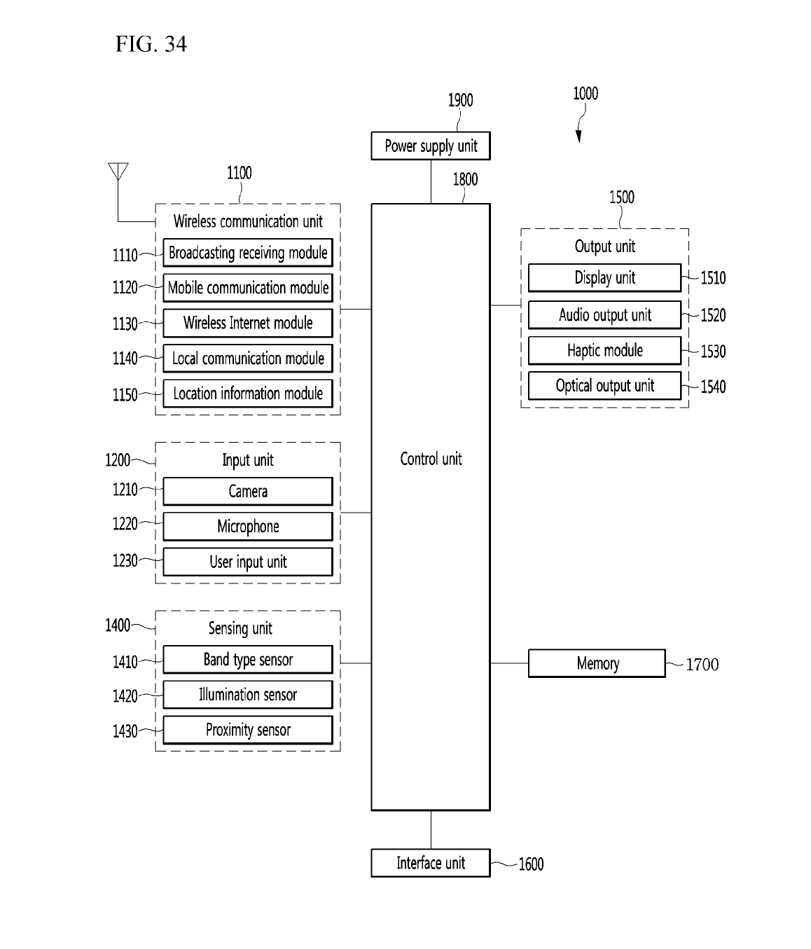

Hereinafter, referring to FIG. 34, each unit included in the wearable device 1000 will be described in more detail.

The wearable device 1000 may include a wireless communication unit 1100, an input unit 1200, a sensing unit 1400, an output unit 1500, an interface unit 1600, a memory unit 1700, a control unit 1800 and a power supply unit 1900. The elements depicted in FIG. 29 are not essential for implementing the wearable device 1000, so the wearable device 1000 according to an embodiment may have elements more than or less than the elements described above.

In more detail, the wireless communication unit 1100 may include at least one module for enabling wireless communication between the wearable device 1000 and the wireless communication system, between the wearable device 1000 and another wearable device 1000, or between the wearable device 1000 and an external server. In addition, the wireless communication unit 1100 may include at least one module for connecting the wearable device 1000 to at least one network.

The wireless communication unit 1100 may include at least one of a broadcasting receiving module 1110, a mobile communication module 1120, a wireless Internet module 1130, a local communication module 1140 and a location information module 1150.

The input unit 1200 may include a camera 1210 or a video input unit for inputting a video signal, a microphone 1220 or an audio input unit for inputting an audio signal, a user input unit 1230, such as a touch key or a mechanical key, for inputting information from a user. The input unit 1200 may process voice or image data collected therein as a control instruction of a user.

The sensing unit 1400 may include at least one sensor for sensing at least one piece of information about an inside of the wearable device 1000, circumference environment surrounding the wearable device 1000 and a user.

Particularly, the sensing unit 1400 may include a band type sensor 1410 according to the embodiment described above. In detail, the sensing unit 1400 may include the gesture sensor of the band type sensor 1410.

In addition, the sensing unit 1400 may further include various sensors. For example, the sensing unit 1400 may include at least one of a proximity sensor 1430, an illumination sensor 1420, an acceleration sensor, a photoplethysmographic sensor, a magnetic sensor, a G-sensor, a gyroscope sensor, an RGB sensor, an infrared (IR) sensor, a finger scan sensor, a ultrasonic sensor, an optical sensor (for example, a camera 1210), a microphone, 1220, a battery gauge, an environment sensor (for example, a barometer, a hygrometer, a thermometer, a radiation sensor, a thermal sensor, a gas sensor, etc.), and a chemical sensor (for example, an electronic nose, a health care sensor, a biometric sensor, etc.). Meanwhile, the wearable device 1000 disclosed through the embodiment may utilize combination of information sensed from at least two of the above sensors.

The output unit may include at least one of a display unit 1510, an audio output unit 1520, a haptic module 1530 and an optical output unit 1540. The display unit 1510 may be combined with a touch sensor in a mutual-layer structure or integrated with a touch sensor to implement a touch screen. The touch screen may be operated as the user input unit 1230 for providing an input interface between the wearable device 1000 and a user and in addition, may provide an output interface between the wearable device 1000 and the user.

The interface unit 1600 may include at least one of a wire/wireless headset port, an external charger port, a wire/wireless data port, a memory cart port, a port for connecting a device having an identification module, an audio I/O (Input/Output) port, a video I/O port and an earphone port. The wearable device 1000 may perform a suitable control related to a connected extern device in response to the connection of the external device to the interface unit 1600.

In addition, the memory 1700 stores data supporting various functions of the wearable device 1000. The memory 1700 may store data and instructions for operations of a plurality of application programs operated in the wearable device 1000, an application, and the wearable device 1000. At least a peace of the application programs may be downloaded from an external server through wireless communication. In addition, at least a piece of the application programs for basic functions of the wearable device 1000 may exist in the wearable device 1000 when the product is released. Meanwhile, the application program is stored in the memory 1700 and installed in the wearable device 1000, so that the operation (or function) of the wearable device 100 may be performed by the control unit 1800.

The control unit controls entire conventional operations of the wearable device 1000 as well as the operation related to the application program. The control unit 1800 may process signals, data and information input or output through the elements described above, or operate the application programs stored in the memory 1700, so that the control unit 1800 may process or provide suitable information or functions to a user.

In addition, the control unit 1800 may control at least one of the elements described in FIG. 29 in order to operate an application program stored in the memory 1700. In addition, the control unit 1800 may combine and operate at least two of the elements included in the wearable device 1000 in order to operate an application program.

The control unit 1800 may control each element of the device according to the user instruction input from the sensing unit 1400. In detail, the control unit 1800 may receive a touch input instruction from the touch sensor to control elements. In addition, the control unit 1800 may receive a gesture input from the gesture sensor to control the elements.

The power supply unit 1900 receives power form an internal power source or an external power source to supply power to each element included in the wearable device 1000 under control of the controller 1800. The power supply unit 1900 may include a battery which is an embedded battery or an exchangeable battery.

At least one of the elements may cooperate with each other and operate to implement an operation, control or a control method of the wearable device 1000 according to various embodiments described below. In addition, the operation, control or control method of the wearable device 1000 may be implemented on the wearable device 1000 by driving at least one application program stored in the memory 1700.

Hereinafter, embodiments related to a method of controlling a wearable device 1000 including a band type sensor 1410 according to the above-described embodiment will be described with reference to accompanying drawings.

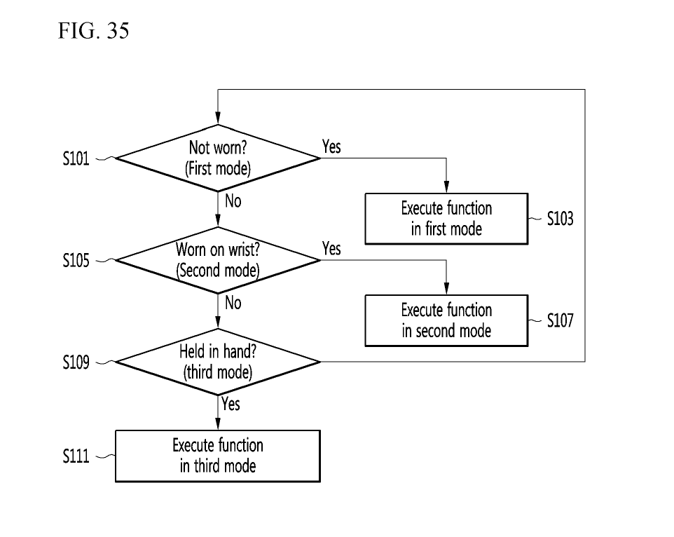

Referring to FIG. 35, in steps S101 and S105, the wearable device 1000 may sense whether a user wears the wearable device 1000.

In detail, the gesture sensor of the wearable device 1000 may sense whether the user makes contact with the band 1005 such that it may be confirmed whether the wearable device 1000 is worn on the user. For example, when the wearing part of a user makes contact with the band in which the gesture sensor is disposed, the gesture sensor may determine whether the user wears the wearable device 1000 based on a variation in capacitance between the sensor electrode of the gesture sensor and the wearing part.

The gesture sensor may transmit the result of sensing whether the wearable device 1000 is worn to the control unit 1800 of the wearable device 1000, and the control 1800 may set a mode of the wearable device 1000 according to whether the wearable device 1000 is worn.

In step S103, when the control 1800 recognizes that the wearable device 1000 is worn, the control 1800 may control such that the wearable device 1000 is set into a first mode.

For example, in the first mode, the control unit 1800 may control the display unit 1510 such that a black image is displayed. In this case, the black image may represent a display off state in which power is not supplied to a screen.

Meanwhile, power is supplied to the touch sensor provided on the display unit 1510, so that the touch sensor is able to sense a touch. Thus, when not worn, power is not supplied to the screen, so that the consumption of power may be reduced.

In addition, when the user does not wear the wearable device 1000, the black image is displayed, and all elements are not activated, but the minimum number of elements, such as the control unit 1800, the sensing unit 140, the input unit 1200, the wireless communication unit 1100 and the output unit 1500 may be activated.

In step S107, when it is determined that the user wears the wearable device 1000, the control unit 1800 of the wearable device 1000 may control the wearable device 1000 in a second mode.

In the second mode, the control unit 1800 may execute entire functions of the wearable device 1000.

For example, a preset image may be displayed on the display unit 1510 in the second mode. The preset image may include a home image including an analog watch image, a digital watch image or a plurality of icons, or simple notice information. Alternatively, the preset image may be a black image or a surrounding image.

The simple notice information may include a numeral, a character, an image or an icon representing a letter message notice, an SNS notice or an E-mail notice. The simple notice information may exclude detail information about each notice such as when, who or what. In order to see the detail information about each notice, after the simple notice information is displayed, an additional event (gesture) must occur or an authorization procedure must be performed.

In the analog watch image, the hands and dials are only displayed in white gradation and the background is displayed in black gradation so that the power may be turned off. In the digital watch image, only digits are displayed in white gradation and the background is displayed in black gradation so that the power may be turned off. Thus, since the power is not supplied for the background occupying most of the analog or digital watch image, the power consumption may be reduced.

Therefore, since the power is rarely consumed even when the analog or digital watch images according to an embodiment are displayed, the analog or digital watch image may not be tuned off but always displayed so far as a user wears the watch type device on the wrist.

Meanwhile, in step S109, the wearable device 1000 may sense whether a user does not wear but holds the wearable device 1000 in his hand.

In detail, when the gesture sensor of the wearable device 1000 recognizes that the wearable device 1000 is not worn and at the same time, the touch sensor senses a touch input, the control unit 1800 may recognize the current mode as the third mode in which the user holds the wearable device 100 in his hand.

When the wearable device 100 is determined to be held in user's hand, the control unit 1800 of the wearable device 1000 may operate the wearable device in the third mode.

For example, when the wearable device 1000 is held, a surrounding image including simple notice information may be displayed on the display unit 1510 of the wearable device 1000. Thus, a user may confirm the simple notice information by holding the wearable device 1000 in his hand even though the wearable device 1000 is not worn on his wrist.

The simple notice information may include a numeral, a character, an image or an icon representing a letter message notice, an SNS notice or an E-mail notice. The simple notice information may exclude detail information about each notice such as when, who or what. In order to see the detail information about each notice, after the simple notice information is displayed, an additional event (gesture) must occur or an authorization procedure must be performed.

Render it down, the wearable device 1000 may determine one of the first to third modes (not worn, worn on wrist and held) and may operate a function in the determined mode.

For example, in the first mode, a black image or a surrounding image may be displayed on the display unit 1510.

For example, in the third mode, an image for a user interface may be displayed on the display unit 1510.

For example, in the second mode, instead of the black image or the surrounding image, a standby image may be displayed on the display unit 1510. The standby image may include an analog watch image, a digital watch image or a plurality of icons.

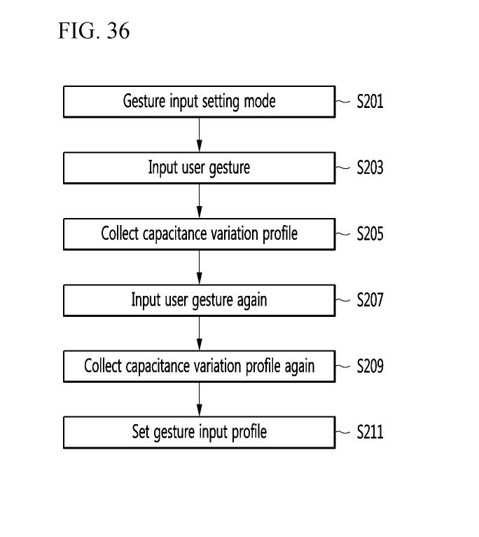

Hereinafter, a method capable of allowing a wearable device 1000 to more precisely sense a gesture of a user by using a gesture sensor will be described with reference to FIG. 36.

As described above, when a user wears the wearable device 1000 including the band type sensor 1410 and makes a gesture, the distance between the wearing part of the user and the band 1005 is varied. In this case, the gesture sensor 1005 may measure the distances between each position of the band 1005 and the wearing part of the user by using the variation in capacitance. Thus, when a user makes a predetermined gesture, the gesture of the user may be sensed by recognizing a variation pattern of the distance between the band 1005 and the wearing part variation.

For example, referring to FIGS. 12 to 17, when a user grasps and opens the hand, the contact area and distance between the wearable device 1000 and the wearing part of the user are changed according to a variation in the human body. In detail, as the muscle of the wrist 10 is contracted when a user grasps his hand, the gesture sensor may be separated from a part of his wrist. For example, the third to fifth electrode patterns 203 to 205 may be away from the wrist. Thus, the third to fifth electrode patterns 203 to 205 may sense that the capacitance coupled with the wrist is reduced.

To the contrary, when a user opens his hand, as the muscle of the wrist expands, the entire gesture sensor may make contact with the wrist. Thus, the first to sixth electrode patterns 201 to 206 may sense that the capacitance coupled with the wrist is great. That is, the sensor electrode 200 may recognize the user gesture by measuring the contact area with the user as described above.

The variation in capacitance may be sensed as a profile. That is, the capacitance sensed during grasping and opening of a hand may be varied in a constant trend.

After storing a history of variations in capacitance according to a gesture (hereinafter, referred to as “capacitance profile”) in the memory, the control unit 1800 compares the capacitance profile with the variation in capacitance input from the gesture sensor to recognize the gesture input of a user.

The capacitance profile may be stored in the memory as default. However, when the capacitance profile is stored as default, it may be difficult to exactly recognize a gesture due to a form of the wearing part of a user or a tightening state of the band 1005.

Thus, the wearable device 1000 may more exactly sense the gesture input of a user through a process of setting a gesture input.

First, the control unit 1800 may provide a gesture input setting mode through the display unit 1510.

A user enters the gesture input setting mode through a touch and thus, may set the entire interface according to a gesture together with the gesture input setting mode.

In step S203, a schedule of a user may be asked through the display in the gesture input setting mode and thus, the user may input a gesture.

In step S205, the gesture sensor may transmit the capacitance profile to the control unit 1800 according to the gesture input of the user.

The control unit 1800 may store the capacitance profile in the memory 1700.

Alternatively, the capacitance profile according to the gesture input of the user may be again collected to set the capacitance profile according to an exact gesture.

Then, in step S211, the control unit 1800 may set a profile according to the gesture input based on the recollected capacitance profile and the initially collected capacitance profile and may store the profile in the memory 1700.

In detail, when the control unit 1800 confirms a matching degree of the recollected capacitance profile and the initially collected capacitance profile so that the matching degree is equal to or more than a preset value, the capacitance profile according to the gesture input may be set based on the recollected capacitance profile and the initially collected capacitance profile.

For example, the control unit 1800 may set the capacitance profile according to the gesture input as an average value of the recollected capacitance profile and the initially collected capacitance profile.

In addition, thereafter, as a user inputs a gesture, the profile information is continuously collected, so that the capacitance profile optimized to the user may be updated.

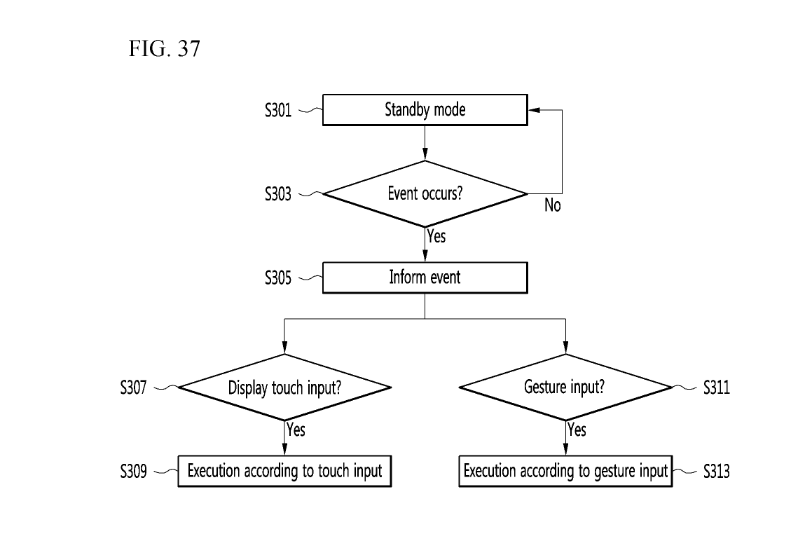

FIG. 37 is a flowchart illustrating an interface using a band type sensor 1410 in a wearable device 1000 according to an embodiment.

Hereinafter, one example of controlling the wearable device 1000 by using the band type sensor 1410 will be described with reference to FIG. 37.

First, in step S301, the wearable device 1000 may wait for a user input in the standby mode.

For example, the wearable device 1000 may stand by in one of the first to third modes described above.

In step S303, a specific event may occur in the wearable device 1000.

For example, a signal for a call may be received through the wireless communication unit of the wearable device 1000.

Next, in step S305, the control unit 1800 may inform a user about the occurrence of an event through at least one element of the output unit.

For example, the display unit 1510 of the output unit may display the contents of an occurring event.

In addition, the audio output unit may output an audio signal such as call signal receiving sound or message receiving sound so that the audio output unit may inform a user about the occurrence of an event. Alternatively, the haptic module of the output unit may inform a user about the occurrence of an event through various tactile sense effects such as a vibration which are sensible by a user.

In addition, a light output unit may output a signal for informing the occurrence of an event by using the light of a light source.

In step S311, the control unit 1800 may receive a gesture input through the band type sensor 1410.

For example, when a call is received through the wearable device 1000, a user may make a gesture of grasping and opening his hand and the band type sensor 1410 may transmit the gesture to the control unit 1800 after recognizing the gesture.

Then, in step S313, the control unit 1800 may execute the operation corresponding to the gesture.

For example, when the gesture of grasping and opening a hand is set to be matched with a signal of allowing the call, the control unit 1800 may recognize the gesture as a signal that the user allows the call, so that the control unit 1800 may control the wireless communication unit to establish the call with a counter party.

Alternatively, if the gesture of grasping and opening a hand is set to be matched with a signal of rejecting the call, the control unit 1800 may reject the call through the wireless communication unit.

In steps S307 and S309, the control unit 1800 may receive a touch input through the band type sensor 1410 and may perform device control according to the touch input.

As described above, the band type sensor 1410 may sense a gesture input as well as a touch input by using a single sensor, so that an interface optimized to the wearable device 1000 may be provided.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

### INDUSTRIAL APPLICABILITY

According to the embodiment provides a band type sensor which is optimized to a wearable device, so that a new interface can be provided.

1. A band type sensor comprising:
a substrate comprising a first active area, a second active area, and an unactive area between the first active area and the second active area;
a touch sensor on the first active area;
a gesture sensor on the second active area; and
a printed circuit board on the unactive area,
wherein the touch sensor and the gesture sensor are connected with the same printed circuit board, and
wherein the gesture sensor senses a gesture of a user in a capacitive type.

2. The band type sensor of claim 1, wherein the gesture sensor includes a sensor electrode and a sensor wiring electrode connected to the gesture sensor.

3. The band type sensor of claim 2, wherein the gesture sensor senses capacitance, which varies depending on a distance between the sensor electrode and a wearing region of the user, to recognize the gesture.

4. The band type sensor of claim 1, wherein the gesture sensor senses variation of a muscle motion of the wearing part according to a gesture input of the user.

5. (canceled)

6. The band type sensor of claim 1, further comprising:
a dummy part interposed between the first and second active areas on the substrate, and
a connecting member disposed on the dummy part.

7. The band type sensor of claim 1, wherein:
the touch sensor detects a touch based on a variation value in capacitance sensed through a sensing electrode, and
the gesture sensor detects a gesture input based on the variation value in capacitance sensed through the sensor electrode.

8. The band type sensor of claim 7, wherein:
the touch sensor senses a position of a touch input through an outer surface in the active area, and
the gesture sensor senses a gesture through an inner surface in the active area.

9. A wearable device comprising:
a main body;
a display part disposed inside the main body;
a band connected to the main body;
a band type sensor including a touch sensor disposed on the display part and a gesture sensor disposed in the band;
a printed circuit board between the display part and the band; and
a control unit to perform an operation according to touch and gesture inputs of a user sensed by the band type sensor,
wherein the touch sensor and the gesture sensor are connected with the same printed circuit board, and
wherein the control unit provides a gesture input setting mode.

10. The wearable device of claim 9, wherein:
the control unit provides a standby mode for allowing a user to input a specific gesture input,
the control unit receives a capacitive profile corresponding to the specific gesture input of the user from the gesture sensor, and
the control unit provides the gesture input setting mode for storing the received capacitive profile.

11. The band type sensor of claim 6, wherein the connecting member includes a magnet.

12. The band type sensor of claim 2, wherein:
the sensor electrode includes a plurality of electrode patterns, and
the electrode patterns are spaced apart from each other.

13. The band type sensor of claim 12, wherein the electrode patterns are disposed to be bilaterally symmetrical with respect to a reference line of the second active area.

14. The band type sensor of claim 2, wherein the sensor electrode senses a gesture of a user through a self-capacitance scheme or a mutual-capacitance scheme.

15. The band type sensor of claim 7, wherein the sensing electrode and the sensor electrode include a same material.

16. The band type sensor of claim 1, wherein the touch and the gesture sensors are disposed on another surface of the substrate.

17. The band type sensor of claim 16, wherein the printed circuit board is disposed on the same surface as the touch sensor.

18. The band type sensor of claim 17, wherein:
the substrate includes a hole, and
the sensor wire electrode is connected to the printed circuit board while passing through the hole of the substrate.

19. The band type sensor of claim 1, wherein the printed circuit board is disposed on one surface of the substrate and opposite surface of the substrate.

20. The band type sensor of claim 19, wherein the printed circuit board is disposed to surround one side surface of the substrate.
